(19) United States
(12) Patent Application Publication
(10) Pub. No.: US 2015/0141264 A1
(43) Pub. Date: May 21, 2015

(54) IN-FIELD DNA EXTRACTION, DETECTION AND AUTHENTICATION METHODS AND SYSTEMS THEREFOR

(71) Applicant: APDN (B.V.I.) Inc., Tortola (VG)

(72) Inventors: Lawrence Jung, Forest Hills, NY (US); MingHwa Benjamin Liang, East Setauket, NY (US)

(21) Appl. No.: 14/471,722

(22) Filed: Aug. 28, 2014

Related U.S. Application Data
(63) Continuation-in-part of application No. 14/210,929, filed on Mar. 14, 2014, now abandoned.
Application No. 14/210,929 is a continuation-in-part of application No. 11/437,265, filed on May 19, 2006, now abandoned.
(60) Provisional application No. 61/794,676, filed on Mar. 15, 2013.
(60) Provisional application No. 60/682,976, filed on May 20, 2005.

(51) Int. Cl. C12Q 1/68

(52) U.S. Cl. CPC C12Q 1/6816; C12Q 1/6844
USPC 506/2; 436/56; 435/6.12; 435/6.11; 506/9; 435/287.2; 422/82.08; 435/7.92; 506/38; 506/39

(57) ABSTRACT

The invention provides a method for in-field detection of a distinctive marker. The method includes providing a sample from an article of interest and analyzing the sample to detect the presence of the distinctive marker. The analysis is performed using an in-field detection instrument. The in-field detection instrument includes a microsystem configured to perform sample in-answer out analysis and detect the presence of the distinctive marker in the sample.

### RELATED APPLICATIONS

This application is a continuation in part of U.S. patent application Ser. No. 14/210,929, filed Mar. 14, 2014, which claims the benefit of U.S. Provisional patent application No. 61/794,676 filed Mar. 15, 2013; this application is also a continuation in part of U.S. patent application Ser. No. 11/437,265, filed on May 19, 2006, which claims the benefit of U.S. Provisional patent application No. 60/682,976, filed on May 20, 2005, the entire disclosures of each of which are incorporated by reference herein in their entireties.

### TECHNICAL FIELD

The invention relates to the use and detection of distinctive markers for tracking and authenticating articles of interest. The distinctive markers can be unique to an object or an article of commerce to be tracked for inventory or security purposes. The invention also relates to detection methods and devices for sampling and detection of such distinctive markers, and the management, storage and processing of identification data obtained by such devices.

### SUMMARY

The present invention provides an instrument, system, and method for in-field detection of a distinctive marker.

According to an exemplary embodiment, the method for in-field detection of a distinctive marker includes providing a sample from an article of interest and analyzing the sample to detect the presence of the distinctive marker. The analysis is performed using an in-field detection instrument. The in-field detection instrument includes a microsystem configured to perform sample in-answer out analysis and detect the presence of the distinctive marker in the sample.

The sample may include a sample identifier, which may include an optical reporter, a digital code, a QR code or a bar code. The optical reporter may include an upconverting phosphor, a fluorophore, an encrypted fluorophore (requiring a developer to reveal the fluorescence), a dye, a stain, a phosphor or a microdot.

The distinctive marker may include a biomolecule or an organic non-biological molecule. The biomolecule may be, for example, an amino acid, a peptide, a protein, a lipid, an isoprene, a monosaccharide, a polysaccharide, a fatty acid, a vitamin, a nucleic acid, a protein-DNA complex, a peptide nucleic acid (PNA), a trace element or isotope.

The distinctive marker may include DNA. The distinctive marker may include one or more unique DNA sequences capable of being copied to produce a unique amplicon profile. The unique amplicon profile may include one or more amplicons of varying length.

The in-field detection instrument may communicate with a server comprising authenticity data for the article of interest.

The sample may be analyzed using PCR, isothermal amplification or nucleic acid hybridization. The sample may be analyzed using next-generation sequencing.

The in-field detection instrument may be portable.

The presence of the distinctive marker may be detected rapidly. For example, the presence of the distinctive marker may be detected substantially instantaneously.

According to an exemplary embodiment, the method includes providing a sample from an article of interest and analyzing the sample to detect the presence of the distinctive marker. The analysis is performed using an in-field detection instrument. The in-field detection instrument includes a microsystem configured to perform sample in-answer out analysis. The authenticity of the article of interest is verified by detecting the presence of the distinctive marker in the sample.

According to an exemplary embodiment, a kit for collecting a sample from an article of interest includes a sample collection unit configured to collect a sample including a distinctive marker suitable for analysis in an in-field detection instrument.

The kit may include a buffer or a solvent suitable for extracting the distinctive marker from an article of interest.

According to an exemplary embodiment, a device for in-field detection of a distinctive marker includes an in-field detection instrument including a microsystem configured to perform sample in-answer out analysis. The in-field detection instrument is configured to analyze a sample to determine the presence of a distinctive marker in the sample.

### BRIEF DESCRIPTION OF THE FIGURES

FIG. 1 is a flow chart of one embodiment of the methods of the invention.

FIG. 2 is a graphical representation of real time PCR results illustrating the detection of a DNA taggant from the fastener in accordance with one embodiment of the methods of the invention.

FIG. 3 is a graphical representation of real time PCR results illustrating the detection of a DNA taggant in accordance with an exemplary embodiment of the methods of the invention.

FIG. 4 shows an embodiment of a process according to the present invention.

FIG. 5 shows an exemplary in-field detection instrument according to an exemplary embodiment of the present invention.

### DETAILED DESCRIPTION

The present invention provides an instrument, a system, and a method for in-field detection of a distinctive marker.

According to an exemplary embodiment, the method for in-field detection of a distinctive marker includes providing a sample from an article of interest and analyzing the sample to detect the presence of the distinctive marker. The analysis is performed using an in-field detection instrument. The in-field detection instrument includes a microsystem configured to perform sample in-answer out analysis and detecting the presence of the distinctive marker in the sample. The authenticity of the article of interest may be verified by detecting the presence of the distinctive marker in the sample.

### Distinctive Markers

The methods of the present invention are for in-field detection of distinctive markers, for example, nucleic acid markers, such as linear or circular, single-stranded or double-stranded DNA, protein or peptide markers and optical reporters and other chemical markers used in the authentication and validation as well as tracking of marked items or articles for security, tracing and anti-counterfeit purposes. A method for tagging and tracing materials with nucleic acids is described in Dollinger (WO 90/14441). The nucleic acid marker can be any suitable linear or circular, single-stranded or double stranded DNA or RNA. The peptide or protein can be any suitable natural or synthetic peptide or protein. The optical reporter can be for instance an upconverting phosphor or a fluorophore. The chemical marker can be any suitable chemical marker, such as, for instance, a dye or a pheromone that can be used for optical or chemical fingerprinting of an item.

A method of forming a detectable fingerprint on an item or article is described in Hatfield (WO 00/55609). DNA fingerprint typing is described in Schulz, M. M., and W. Reichert. “Archived or directly swabbed latent fingerprints as a DNA source for STR typing” Forensic science international 127.1 (2002): 128-130.

A distinctive marker may be any detectable marker suitable for distinguishing a unique article from other articles. The distinctive marker may be a nucleic acid (e.g., DNA), a protein, a peptide, an optical reporter or a marker chemical. For example, the distinctive marker may include one or more nucleic acids having one or more unique nucleic acid sequences. The distinctive marker may include a unique combination of two or more nucleic acid sequences. The distinctive marker may include a combination of nucleic acid sequences that, when copied, produce a unique combination of amplicons having particular lengths. That is, the combination of amplicons produce a unique variable amplicon length profile. The nucleic acids of the distinctive marker may include DNA.

The distinctive marker may include a biomolecule or an organic non-biological molecule. A biomolecule is a molecule produced by living organisms. The biomolecule may be an amino acid, a peptide, a protein, a lipid, an isoprene, a monosaccharide, a polysaccharide, a fatty acid, a vitamin, a nucleic acid, a protein-DNA complex, or a peptide nucleic acid (PNA).

The distinctive marker may include DNA. The distinctive marker may include one or more unique DNA sequences capable of being copied to produce a unique amplicon profile. For example, a unique amplicon profile may include a particular combination of one or more nucleic acid sequences that is unique to a particular article. The one or more DNA sequences themselves may be unique or the combination of DNA sequences may be unique to the article. The unique amplicon profile may include one or more amplicons of varying length. For example, the distinctive marker may include a combination of nucleic acid sequences that, when copied, produce a unique combination of amplicons having a particular combination of lengths. That is, the combination of amplicons produce a unique variable amplicon length profile. According to exemplary embodiments, the distinctive marker may include a unique combination of DNA sequences capable of being copied to produce a unique combination of amplicons including both distinctive nucleic acid sequences, and distinctive amplicon lengths.

The nucleic acid marker may be synthetically produced using a nucleic acid synthesizer or by isolating nucleic acid material from yeast, human cell lines, bacteria, animals, plants and the like. In exemplary embodiments, the nucleic acid material may be treated with restriction enzymes and then purified to produce an acceptable nucleic acid marker(s). A method of nucleic acid code analysis is described in Guo (WO 98/06084). The nucleic acid taggant may comprise one specific nucleic acid sequence or alternatively, may comprise a plurality of various nucleic acid sequences. In one embodiment, polymorphic DNA fragments including short tandem repeats (STRs) revealed as fragment length polymorphisms and/or single nucleotide polymorphisms (SNP) are utilized as an anti-counterfeit nucleic acid tag. While the use of a single sequence for a nucleic acid marker may make detection of the marker easier and quicker, the use of a plurality of nucleic acid sequences including STR and SNP, in general, may provide a higher degree of security against forgers.

In exemplary embodiments of the methods of the invention, the nucleic acid marker is derived from DNA extracted from a specific plant source and is specifically digested and ligated to generate artificial nucleic acid sequences which are unique to the world. The digestion and ligation of the extracted DNA is completed by standard restriction digestion and ligation techniques known to those skilled in the art of molecular biology. Once the modified DNA taggant has been produced, the taggant may be encapsulated into materials for protection against UV light and/or degradation. The DNA encapsulant materials may be of plant origin. The sequence of the DNA marker can be of any suitable length, for instance the sequence of the DNA marker can be a sequence of from about 20 to about 1000 bases.

After the nucleic acid fragment with a known nucleic acid sequence has been manufactured or isolated, the method further comprises, producing a DNA marker compound which comprises the selected nucleic acid fragment. The marker compound may be produced as a solid or liquid, water or oil based, a suspension, an aggregate and the like. A feature of the marker compound may be to protect the nucleic acid fragment from UV light and/or other degradation factors that may degrade the nucleic acid taggant over time, while the nucleic acid is acting as an authentication tag for a particular product. In exemplary embodiments, when the taggant is DNA, the nucleic acid tag may be encapsulated and suspended in a solvent solution (e.g., an aqueous or an organic solvent solution) producing a “stock” DNA taggant solution at a specified concentration. The stock DNA solution can be added to the marker compound mixture at an appropriate concentration for the type of product to be authenticated. In exemplary embodiments, the DNA taggant may be mixed with other components of the marker compound without any prior encapsulation. Processes such as nucleic acid fragment encapsulation and other techniques utilized for protecting nucleotides, and in particular, DNA from degradation are described more fully below.

Another feature of the marker compound mixture is to be able to camouflage or “hide” the specified nucleic acid tag with extraneous and nonspecific nucleic acid oligomers/fragments, thus making it difficult for unauthorized individuals, such as forgers to identify the sequence of the nucleic acid tag. In exemplary embodiments, the marker compound comprises a specified dsDNA taggant from a known source (i.e. mammal, invertebrate, plant and the like) along with genomic DNA from the corresponding or similar DNA source. The amount of the DNA taggant found in a marker compound varies depending on the particular product to be authenticated, the duration the taggant needs to be viable (e.g. 1 day, 1 month, 1 year, multiple years) prior to authentication, expected environmental exposure, the detection method to be utilized, etc.

According to exemplary embodiments, the taggant is dsDNA, and PCR is the technique for taggant detection. The copy number of DNA taggant in a predetermined sample size of marker compound used for authentication is about 3 copies to about 100,000 copies, for example, about 10 copies to about 50,000 copies, or about 100 copies to about 10,000 copies of DNA taggant.

### Incorporation of Functional Groups

In exemplary embodiments, the nucleic acid tag is labeled with at least one compound or “detection molecule” prior to being incorporated into the specified product to aid in the extraction and/or detection of the nucleic acid marker from the product after being placed in a supply chain. A detection molecule is a molecule or compound with at least one detectable moiety. For example, fluorescent molecules may be configured to be combined with the nucleic acid marker for certain detection methods which are described in detail below.

In exemplary embodiments of the present invention, suitable dyes include, but are not limited to, coumarin dyes, xanthene dyes, resorufins, cyanine dyes, difluoroboradiazaindacene dyes (BODIPY), ALEXA dyes, indoles, bimanes, isoindoles, dansyl dyes, naphthalimides, phthalimides, xanthenes, lanthanide dyes, rhodamines and fluoresceins. Certain visible and near IR dyes are known to be sufficiently fluorescent and photostable to be detected as single molecules. In this aspect the visible dye, BODIPY R6G (525/545), and a larger dye, LI-COR's near-infrared dye, IRD-38 (780/810) can be detected with single-molecule sensitivity and may be used to practice the authentication process described herein. In exemplary embodiments of the present invention, suitable dyes include, but are not limited to, fluorescein, 5-carboxyfluorescein (FAM), rhodamine, 5-(2′-aminoethyl) aminonapthalene-1-sulfonic acid (EDANS), anthranilamide, coumarin, terbium chelate derivatives, Reactive Red 4, BODIPY dyes and cyanine dyes.

There are many linking moieties and methodologies for attaching fluorophore or visible dye moieties to nucleotides, as exemplified by the following references: Eckstein, editor, Oligonucleotides and Analogues: A Practical Approach (IRL Press, Oxford, 1991); Zuckerman et al., Nucleic Acids Research, 15: 5305-5321 (1987) (3′ thiol group on oligonucleotide); Sharma et al., Nucleic Acids Research, 19: 3019 (1991) (3′ sulfhydryl); Giusti et al., PCR Methods and Applications, 2: 223-227 (1993) and Fung et al., U.S. Pat. No. 4,757,141 (5′ phosphoamino group via Aminolink™ II available from Applied Biosystems, Foster City, Calif.) Stabinsky, U.S. Pat. No. 4,739,044 (3′ aminoalkylphosphoryl group); AP3 Labeling Technology (U.S. Pat. Nos. 5,047,519 and 5,151,507, assigned to E.I. DuPont de Nemours & Co); Agrawal et al, Tetrahedron Letters, 31: 1543-1546 (1990) (attachment via phosphoramidate linkages); Sproat et al., Nucleic Acids Research, 15: 4837 (1987) (5′ mercapto group); Nelson et al, Nucleic Acids Research, 17: 7187-7194 (1989) (3′ amino group); and the like.

In exemplary embodiments, the complementary nucleic acid probe is labeled with at least one compound or molecule with functionality to aid in the detection of the nucleic acid tag/marker. The techniques and dyes utilized in labeling the nucleic acid tag or the complementary probe may be the same due to the nucleic acid being combined with the probe.

The detection molecules of the invention can be incorporated into probe motifs, such as Taqman probes (Held et al., Genome Res. 6: 986-994 (1996), Holland et al., Proc. Nat. Acad. Sci. USA 88: 7276-7280 (1991), Lee et al., Nucleic Acids Res. 21: 3761-3766 (1993)), molecular beacons; Tyagi et al., Nature Biotechnol., 16:49-53 (1998), U.S. Pat. No. 5,989,823, issued Nov. 23, 1999)) scorpion probes (Whitcomb et al., Nature Biotechnology 17: 804-807 (1999)), sunrise probes (Nazarenko et al., Nucleic Acids Res. 25: 2516-2521 (1997)), conformationally assisted probes (Cook, R., copending and commonly assigned U.S. Provisional Application No. 60/138,376, filed Jun. 9, 1999), peptide nucleic acid (PNA)-based light up probes (Kubista et al., WO 97/45539, December 1997), double-strand specific DNA dyes (Higuchi et al. Bio/Technology 10: 413-417 (1992), Wittwer et al, Bio/Techniques 22: 130-138 (1997)) and the like. These and other probe motifs with which the present detection molecules can be used are reviewed in Nonisotopic DNA Probe Techniques, Academic Press, Inc. 1992.

In exemplary embodiments, the molecular beacon system is utilized to detect and quantify the nucleic acid tag from the product of interest. “Molecular beacons” are hairpin-shaped nucleic acid detection probes that undergo a conformational transition when they bind to their target that enables the molecular beacons to be detected. In general, the loop portion of a molecular beacon is a probe nucleic acid sequence which is complementary to the nucleic acid marker. The stem portion of the molecular beacon is formed by the annealing of arm sequences of the molecular beacon that are present on either side of the probe sequence. A functional group such as a fluorophore (e.g. coumarin, EDNAS, fluorescein, lucifer yellow, tetramethylrhodamine, texas red and the like) is covalently attached to the end of one arm and a quencher molecule such as a nonfluorescent quencher (e.g. DABCYL) is covalently attached to the end of the other arm. When there is no target (nucleic acid tag) present, the stem of the molecular beacon keeps the functional group quenched due to its close proximity to the quencher molecule. However, when the molecular beacon binds to its specified target, a conformational change occurs to the molecular beacon such that the stem and loop structure cannot be formed, thus increasing the distance between the functional group and the quencher which enables the presence of the target to be detected. When the functional group is a fluorophore, the binding of the molecular beacon to the nucleic acid tag is detected by fluorescence spectroscopy.

In exemplary embodiments, a plurality of nucleic acid tags with varying sequences are used in labeling a particular product. The different nucleic acid tags can be detected quantitatively by a plurality of molecular beacons, each with a different colored fluorophore and with a unique probe sequence complementary to at least one of the plurality of nucleic acid tags. Being able to quantitate the various fluorophores (i.e. various nucleic acid tags) provides a higher level of authentication and security. It should be noted, that the other functional groups described above useful in labeling nucleic acid probes can also be utilized in molecular beacons for the present invention.

### Application of the Distinctive Marker to an Article

According to exemplary embodiments, the item to be marked can be any suitable item, such as for instance a microchip, a label, a badge, a logo, a printed material, a document, a textile or a commodity. Paper labeled with biochemical markers is described in Wiggins (WO 02/057548). In one embodiment, the invention provides a method for in-field detection and authentication of a distinctive marker, such as, for instance, DNA, a protein, a peptide an optical reporter and a marker chemical. According to an exemplary embodiment, the method includes providing a sample from an item of interest; analyzing the sample to detect presence of the distinctive marker encoding information unique to said item using a specific detection method tailored to the distinctive marker, wherein the detection method is performed using a portable device; and verifying the authenticity of the item by determining that the distinctive marker is present in the analyzed sample.

In exemplary embodiments, the DNA marked item that can be authenticated by the methods of the invention can be any suitable item, such as a high value item or a unique item, or an item having a critical function. The marked item can be an item of electronics, such as for instance a microchip; alternatively, the item can be an item of household goods, fabrics, textiles, labels, badges, logos, or items of clothing or accessories. In an exemplary embodiment, the marked item can be an ink, documents or printed material, or a commodity. In an exemplary embodiment, the marked item can be a pharmaceutical product, such as, for instance, and without limitation, a tablet, a caplet, a powder or a lozenge. In one embodiment, the item is selected from the group consisting of cash, a currency note, a coin, a gem an item of jewelry, a musical instrument, a passport, an antique, an item of furniture, artwork, a property deed, a stock certificate, or a bond certificate. The marked item can be an inventory item or non-inventoried item in transit.

In one embodiment, the DNA marked item that can be authenticated by the methods of the invention is a microchip, a label, a badge, a logo, a printed material, a textile or a commodity. In an exemplary embodiment, the item is selected from the group consisting of cash, a currency note, a coin, a gem an item of jewelry, a musical instrument, a passport, an antique, an item of furniture, artwork, a property deed, a stock certificate, or a bond certificate.

The distinctive marker may be applied to an article of interest by any available means suitable for securing the distinctive marker to the article of interest, such that the distinctive marker will not be easily removed while being moved through the stream of commerce or through deliberate tampering, so that a sample of the distinctive marker may be acquired from the article of interest at any point in the stream of commerce. The terms “article,” “item,” and “article of interest” may be used interchangeably throughout the specification and drawings. The terms “marker,” “distinctive marker,” “security marker,” “tag” and “taggant” may be used interchangeably throughout the specification and drawings. A sample may include any partial or complete capturing of a portion of the distinctive marker from the article of interest having the distinctive marker coupled thereto. For example, the sample of the distinctive marker may be captured by using any suitable mechanical or chemical means. Multiple samples may be taken of the same distinctive marker on the same article of interest at multiple points along the stream of commerce and/or at different times as the article moves through the stream of commerce. Multiple iterations of the same distinctive marker may be placed at multiple locations on the article of interest or multiple iterations of different distinctive markers may be placed on the article of interest. For example, the distinctive marker may be placed at one location on the article of interest, but a non-distinctive marker (e.g., a camouflage or decoy marker) may also be placed on the same article of interest.

An article of interest may be any item, product, object, liquid, or gas, such as, for instance, a microchip, a label, a badge, a logo, a printed material, a textile, a commodity, an ink or a solution.

The article of interest may be marked with a relatively small amount of the distinctive marker such that the location of the distinctive marker on the article of interest is not readily visible to the naked eye, and/or not readily detectable through tactile inspection of the article. However, the amount of distinctive marker is not limited thereto, and any desired amount of distinctive marker may be used to mark an article. For example, a relatively large amount of distinctive marker may be applied to the article, and it may be desirable for the distinctive marker to not be concealed. A marking apparatus for nucleic acid marking of items is described in Sleat (WO 03/080931). A method of applying a DNA sample to an item is described in Mackay (GB 2434570 A). A method of marking a solid article is described in Slater (WO 95/02702). A method of marking a product for identification is described in Garner, et al. (WO 95/06249). A means of labeling an item or substance is described in Le Page, et al. (WO 87/06383). A process for labeling an article and/or document is described in Regan, et al. (WO 03/030129).

The method of incorporating the nucleic acid tag into a product may depend on the type of product to be authenticated, as described above. The nucleic acid tag may be added to a marker compound in a “naked” or encapsulated form at a predetermined concentration, which allows for accurate detection of the nucleic acid taggant. The marker compound may be a liquid but in exemplary embodiments may be a solid. The marker compound may be a liquid and after the addition of the nucleic acid taggant, may be dried prior to introducing the marker onto a particular product (e.g. a drug tablet, or textile). When the marker compound comprising a nucleic acid taggant is in liquid form, the marker compound may be applied to the product in a lacquer, paint or liquid aerosol form.

In one embodiment, the microfluidic device of the invention employs an enzymatic process to degrade biological molecules such as proteins and lipids in the sample, such as a saliva sample or a swab from a cheek smear, and to release the DNA content for downstream analysis and detection. The released DNA then flows in the microfluidic channels to a PCR thermocycler module for amplification and detection by another module such as a capillary electrophoresis module. Detection can be by any number of methods, such as optical, radioactivity detection, ISFET or any of the methods known to those of skill in the art of oligonucleotide sequencing and detection methods. The data generated can be analyzed in the module or sent via a secure server to a network server or to a server in the cloud for comparison with other stored data or for further analysis. Such systems can be programmed to give data readouts in 90 minutes or less. One application of such methods is for biometrics for genealogy or for identity verification of personnel or travelers etc.

### Sample Identifier

In one embodiment, the DNA marker is associated with a sample identifier (e.g., detectable reporter) and the method further includes locating the optical reporter associated with the DNA marker thereby facilitating sampling the DNA marker for the in-field detection and authentication procedure. The sample identifier may be an optical reporter, a digital code, a QR code or a bar code. The optical reporter may include an upconverting phosphor, a florophore, a dye, a stain, or a phosphor. In an exemplary embodiment, the detectable reporter is selected from a chemical reporter, a digital reporter and/or a peptide reporter. In an exemplary embodiment, the digital reporter can be any suitable digital reporter, such as, for instance, a barcode, a QR code, a quantum dot, or an RFID. In another embodiment the QR code, quantum dot, bar code or RFID may include the encrypted sequence of the DNA marker, see for instance: Tran et al., Verification or physical encryption taggants using digital representatives and authentications thereof, WO 2013/170009.

A sample identifier may be combined with the distinctive marker. The sample identifier may be any suitable sample identifier to assist in locating the distinctive marker. For example, the sample identifier may be used to identify the location of the distinctive marker which has been coupled to the article of interest. The sample identifier may not be readily detectible by visual or tactile inspection of the article of interest, similar to the distinctive marker. For example, the sample identifier may be an optical reporter, such as a fluorescent dye, which is only activated upon exposure to UV light. Thus, exposure of the article of interest having the distinctive marker combined with the optical reporter may visually reveal the location of the distinctive marker on the article of interest. A method and ink set for marking articles is described in Degott (EP 1403333 A1).

The sample identifier may be a tactile identifier, such as a course pattern, and may be disposed on the surface of the article of interest at the location where the distinctive marker is located. The tactile identifier may be an unusually smooth pattern formed on the surface of the article of interest. However, the distinctive marker may be applied to the article without the sample identifier.

The DNA marker itself may be a detectable DNA marker. The detectable DNA marker may be labeled, for example, with a fluorescent label or probe.

### Sampling of the Marker

The sample for in-field detection and authentication of a DNA marker can be any suitable sample, such as for instance a solid sample from a surface, a scraping, a cutting, a powder, a liquid, a mist, or a swab. The sample can be collected in a collection vessel, such as a plastic or glass test tube, a screw-cap tube, a microcap tube, a well of an assay plate or a microfluidic chamber, reservoir or other suitable container.

A sample including some, or all, of the distinctive marker may be collected from the article of interest and analyzed to detect the presence of the distinctive marker. The sample may be analyzed by any suitable means for detecting the presence of the distinctive marker. For example, the analysis may be performed using an in-field detection instrument. The in-field detection instrument is an instrument that can complete an analysis without the need for transporting a sample to a lab. For example, the sample of the distinctive marker may be taken from a marked product before shipping the product from a factory to a consumer, and the sample may be analyzed by the in-field detection instrument in the factory, without the need to transport the sample to a lab for testing. Thus, the analysis may be performed rapidly, either substantially instantaneously or within minutes of collecting the sample. For example, the presence of the distinctive marker may be detected substantially instantaneously (e.g., within 10-20 seconds), in real-time, or within minutes of transferring the sample to the in-field detection instrument. For example, the analysis and/or detection of the distinctive marker may be completed in about 5 minutes to about 2 hours. More particularly, the analysis and/or detection of the distinctive marker may be completed in about 10 minutes to about 30 minutes. The authenticity of the article of interest may be verified by detecting the presence of the distinctive marker in the sample.

According to an exemplary embodiment, a kit for collecting a sample from an article of interest includes a sample collection unit configured to collect a sample including a distinctive marker suitable for analysis in an in-field detection instrument. A variety of nucleic acid extraction solutions (e.g., solvents or buffers) have been developed for extracting nucleic acid sequences from a sample of interest. For instance, nucleic acid extraction procedures are described in Green, Michael R., and Joseph Sambrook. Molecular cloning: a laboratory manual. New York: Cold Spring Harbor Laboratory Press, 2012. In an embodiment, the sample collection kit may include a buffer or a solvent suitable for extracting the distinctive marker from an article of interest that is compatible with the analysis processes carried out by the in-field detection instrument.

### Nucleic Acid Tag Extraction and Capture Methods

A variety of nucleic acid extraction solutions have been developed over the years for extracting nucleic acid sequences from a sample of interest. See, for example, Green, Michael R., and Joseph Sambrook. Molecular cloning: a laboratory manual. New York: Cold Spring Harbor Laboratory Press, 2012. Many such methods typically require one or more steps of, for example, a detergent-mediated step, a proteinase treatment step, a phenol and/or chloroform extraction step, and/or an alcohol precipitation step. Some nucleic acid extraction solutions may comprise an ethylene glycol-type reagent or an ethylene glycol derivative to increase the efficiency of nucleic acid extraction while other methods only use grinding and/or boiling the sample in water. Other methods, including solvent-based systems and sonication, could also be utilized in conjunction with other extraction methods.

In exemplary embodiments, the authentication process comprises capturing the nucleic acid tag directly with a complementary hybridization probe attached to a solid support. In general, the methods for capturing the nucleic acid tag involve a material in a solid-phase interacting with reagents in the liquid phase. In exemplary embodiments, the nucleic acid probe is attached to the solid phase. The nucleic acid probe can be in the solid phase such as immobilized on a solid support, through any one of a variety of well-known covalent linkages or non-covalent interactions. In exemplary embodiments, the support is comprised of insoluble materials, such as controlled pore glass, a glass plate or slide, polystyrene, acrylamide gel and activated dextran. In exemplary embodiments, the support has a rigid or semi-rigid character, and can be any shape, e.g. spherical, as in beads, rectangular, irregular particles, gels, microspheres, or substantially flat support. In exemplary embodiments, it can be desirable to create an array of physically separate sequencing regions on the support with, for example, wells, raised regions, dimples, pins, trenches, rods, pins, inner or outer walls of cylinders, and the like. Other suitable support materials include, but are not limited to, agarose, polyacrylamide, polystyrene, polyacrylate, hydroxethylmethacrylate, polyamide, polyethylene, polyethyleneoxy, or copolymers and grafts of such. Exemplary solid-supports include small particles, non-porous surfaces, addressable arrays, vectors, plasmids, or polynucleotide-immobilizing media.

As used in the methods of capturing the nucleic acid tag, a nucleic acid probe can be attached to the solid support by covalent bonds, or other affinity interactions, to chemically reactive functionality on the solid-supports. The nucleic acid can be attached to solid-supports at their 3′, 5′, sugar, or nucleobase sites. In exemplary embodiments, the 3′ site for attachment via a linker to the support is preferred due to the many options available for stable or selectively cleavable linkers. Immobilization is preferably accomplished by a covalent linkage between the support and the nucleic acid. The linkage unit, or linker, is designed to be stable and facilitate accessibility of the immobilized nucleic acid to its sequence complement. Alternatively, non-covalent linkages such as between biotin and avidin or streptavidin are useful. Examples of other functional group linkers include ester, amide, carbamate, urea, sulfonate, ether, and thioester. A 5′ or 3′ biotinylated nucleotide can be immobilized on avidin or streptavidin bound to a support such as glass.

Depending on the initial concentration of the nucleic acid tag added to the product of interest, the tag can be detected quantitatively without being amplified by PCR. In exemplary embodiments, a single stranded DNA tag labeled with a detection molecule (i.e. fluorophore, biotin, etc.) can be hybridized to a complementary probe attached to a solid support to allow for the specific detection of the “detection molecule” configured to the tag. The nucleic acid DNA tag can also be double stranded, with at least one strand being labeled with a detection molecule. With a dsDNA tag, the nucleic acid tag must be heated sufficiently and then quick cooled to produce single stranded DNA, where at least one of the strands configured with a detection molecule is capable of hybridizing to the complementary DNA probe under appropriate hybridization conditions.

In exemplary embodiments of the present invention, the complementary probe is labeled with a detection molecule and allowed to hybridize to a strand of the nucleic acid tag. The hybridization of the probe can be completed within the product, when the product is, for example, a textile, or can be completed after the nucleic acid tag/marker has been extracted from the product, such as when the products are liquid (e.g. oil, gasoline, perfume, etc.). The direct detection methods described herein may depend on having a large initial concentration of nucleic acid label embedded into the product or rigorous extraction/capture methods which concentrate the nucleic acid tag extracted from a large volume or mass of a particular product.

The technology has made possible the sequencing of the entire genome of a person (e.g., whole genome amplification) to identify differences in the genetic code among individuals. It was discovered that about one base in every thousand of each of us differs in one individual from the next. These differences are called genetic variations or single nucleotide polymorphisms (SNP). These variations have been widely explored in the medical field for personalized medicine and to determine whether an individual is predisposed to diseases such as diabetes.

### Sample Analysis & Marker Detection

The sample may be analyzed, for example, using PCR, isothermal amplification or nucleic acid hybridization. The sample may be analyzed using next-generation sequencing. Methods of sample analysis are described in more detail below.

### DNA Amplification

In exemplary embodiments, the authentication process comprises amplifying the nucleic acid marker by polymerase chain reaction. During amplification, primer dimers and other extraneous nucleic acids may be amplified together with the nucleic acid corresponding to the analyte. These impurities may be separated, for example, by gel separation techniques, from the amplified product. To allow an amount of PCR product to form which is sufficient for later analysis, quantitative competitive PCR amplification uses an internal control competitor and is stopped after a log phase of product formation has been completed.

In exemplary embodiments, PCR amplification is performed in the presence of a non-primer detectable probe which specifically binds the PCR amplification product, i.e., the amplified detector DNA moiety. PCR primers may be designed according to known criteria and PCR may be conducted in commercially available instruments. The probe may be a DNA oligonucleotide specifically designed to bind to the amplified detector molecule. The probe preferably has a 5′ reporter dye and a downstream 3′ quencher dye covalently bonded to the probe which allow fluorescent resonance energy transfer. Suitable fluorescent reporter dyes include 6-carboxy-fluorescein (FAM), tetrachloro-6-carboxy-fluorescein (TET), 2,7-dimethoxy-4,5-dichloro-6-carboxy-fluorescein (JOE) and hexachloro-6-carboxy-fluorescein (HEX). A suitable reporter dye is 6-carboxy-tetramethyl-rhodamine (TAMRA). These dyes are commercially available from Perkin-Elmer, Philadelphia, Pa. Detection of the PCR amplification product may occur at each PCR amplification cycle. At any given cycle during the PCR amplification, the amount of PCR product is proportional to the initial number of template copies. The number of template copies is detectable by fluorescence of the reporter dye. When the probe is intact, the reporter dye is in proximity to the quencher dye which suppresses the reporter fluorescence. During PCR, the DNA polymerase cleaves the probe in the 5′-3′ direction separating the reporter dye from the quencher dye increasing the fluorescence of the reporter dye which is no longer in proximity to the quencher dye. The increase in fluorescence is measured and is directly proportional to the amplification during PCR. This detection system is now commercially available as the TaqMan® PCR system from Perkin-Elmer, which allows real time PCR detection. Probe analysis of nucleic acids is described in Kubista, Mikael, et al. (WO 97/45539). PCR product detection using self-probing amplicons is described in Whitcombe, David, et al. “Detection of FOR products using self-probing amplicons and fluorescence” Nature biotechnology 17.8 (1999): 804-807. Oligonucleotide probes synthesized using controlled pore glass (CPG) is described in Nelson, Paul S., Roy A. Frye, and Edison Liu. “Bifunctional oligonucleotide probes synthesized using a novel CPG support are able to detect single base pair mutations” Nucleic acids research 17.18 (1989): 7187-7194. Fluorogenic probes used in nick-translation FOR are described in Lee, Linda G., Charles R. Connell, and Will Bloch. “Allelic discrimination by nick-translation PCR with fluorgenic probes” Nucleic acids research 21.16 (1993): 3761-3766.

In an exemplary embodiment, the reporter dye and quencher dye may be located on two separate probes which hybridize to the amplified PCR detector molecule in adjacent locations sufficiently close to allow the quencher dye to quench the fluorescence signal of the reporter dye. As with the detection system described above, the 5′-3′ nuclease activity of the polymerase cleaves the one dye from the probe containing it, separating the reporter dye from the quencher dye located on the adjacent probe preventing quenching of the reporter dye. As in the embodiment described above, detection of the PCR product is by measurement of the increase in fluorescence of the reporter dye.

Real time quantitative PCR has been applied to nucleic acid analytes or templates. In this method, PCR is used to amplify DNA in a sample in the presence of a nonextendable dual labeled fluorogenic hybridization probe. One fluorescent dye serves as a reporter and its emission spectra is quenched by the second fluorescent dye. The method uses the 5′ nuclease activity of Taq polymerase to cleave a hybridization probe during the extension phase of PCR. The nuclease degradation of the hybridization probe releases the quenching of the reporter dye resulting in an increase in peak emission from the reporter. The reactions are monitored in real time. Reverse transcriptase PCR (RT-PCR) and Real-Time PCR have also been described in Heid. Christian A., et al. “Real time quantitative PCR” Genome research 6.10 (1996): 986-994: and Gibson, U. E., Christian A. Heid, and P. Mickey Williams. “A novel method for real time quantitative RT-PCR” Genome research 6.10 (1996): 995-1001. Numerous commercial thermal cyclers are available that can monitor fluorescent spectra of multiple samples continuously in the PCR reaction, therefore the accumulation of PCR product can be monitored in ‘real time’ without the risk of amplicon contamination of the laboratory. Heid, C. A.; Stevens, J.; Livak, K. L.; Williams, P. W. (1996). Real time quantitative PCR. Gen. Meth. 6: 986-994.

In exemplary embodiments, real time PCR detection strategies may be used; including known techniques such as visualization with intercalating dyes (e.g., ethidium bromide) and other double stranded DNA binding dyes used for detection (e.g. SYBR green, a highly sensitive fluorescent stain, FMC Bioproducts), dual fluorescent probes (described in Wittwer, et al., (1997) BioTechniques 22: 176-181) and panhandle fluorescent probes (i.e. molecular beacons; described in Tyagi, and Kramer (1996) Nature Biotechnology 14: 303-308). Although intercalating dyes and double stranded DNA binding dyes permit quantitation of PCR product accumulation in real time applications, they may lack specificity, detecting primer dimer and any non-specific amplification product. Careful sample preparation and handling, as well as careful primer design, using known techniques may minimize the presence of matrix and contaminant DNA and prevent primer dimer formation. Appropriate PCR instrument analysis software and melting temperature analysis provide a means to extract specificity and may be used with these embodiments.

Molecular beacons systems may be used with real time PCR for specifically detecting the nucleic acid template in the sample quantitatively. For instance, the commercially available Roche Light Cycler™ (Roche Diagnostics Corporation, Indianapolis, Ind.). or other such instruments may be used for this purpose. The detection molecule configured to the molecular beacon probe may be visible under daylight or conventional lighting and/or may be fluorescent. It should also be noted that the detection molecule may be an emitter of radiation, such as a characteristic isotope. Multicolor molecular beacons are described in Tyagi, Sanjay, Diana P. Bratu, and Fred Russell Kramer. “Multicolor molecular beacons for allele discrimination” Nature biotechnology 16.1 (1998): 49-53. Fluorescent molecular beacons are described in Tyagi, Sanjay, and Fred Russell Kramer. “Molecular beacons: probes that fluoresce upon hybridization” Nature biotechnology 14.3 (1996): 303-308.

In one embodiment, DNA may be amplified by nanopore sequencing. Nanopore sequencing includes using a small aperture (e.g., a nanopore of approximately 1 nanometer in diameter) immersed in a conducting fluid with a voltage applied across the nanopore. When individual nucleotides pass through or near the nanopore a measurable change in voltage may occur, which can be used to identify individual nucleotides of a DNA strand. For instance, nanopore sequencing is described in Branton, Daniel, et al. “The potential and challenges of nanopore sequencing” Nature biotechnology 26.10 (2008): 1146-1153; and Deamer, David W., and Mark Akeson. “Nanopores and nucleic acids: prospects for ultrarapid sequencing” Trends in biotechnology 18.4 (2000): 147-151.

In one embodiment, DNA may be amplified by Multiple Annealing and Looping Based Amplification Cycles (MALBAC). MALBAC may be used to amplify substantially a whole genome. MALBAC may amplify a genome in quasi-linear fashion and may be used for single cell, whole genome amplification. In MALBAC, amplicons may have complementary ends. The complementary ends may form loops, which may prevent exponential amplicon copying, thus preventing amplification bias. For instance, MALBAC is described in Zong, Chenghang, et al. “Genome-wide detection of single-nucleotide and copy-number variations of a single human cell” Science 338.6114 (2012): 1622-1626.

The ability to rapidly and accurately detect and quantify biologically relevant molecules with high sensitivity is a central issue for medical technology, national security, public safety, and civilian and military medical diagnostics. Many of the currently used approaches, including enzyme linked immunosorbant assays (ELISAs) and PCR are highly sensitive. However, the need for PCR amplification may make a detection method more complex, costly and time-consuming. In exemplary embodiments anti-counterfeit nucleic acid tags are detected by Surface Enhanced Raman Scattering (SERS) as described in Graham et al. (U.S. Pat. No. 6,127,120). SERS is a detection method which is sensitive to relatively low target (nucleic acid) concentrations, which can preferably be carried out directly on an unamplified sample. Nucleic acid tags and/or nucleic acid probes can be labeled or modified to achieve changes in SERS of the nucleic acid tag when the probe is hybridized to the nucleic acid tag. The use of SERS for quantitatively detecting a nucleic acid provides a relatively fast method of analyzing and authenticating a particular product.

Another detection method useful in the invention is the Quencher-Tether-Ligand (QTL) system for a fluorescent biosensor described in Whitten et al. (U.S. Pat. No. 6,743,640). The QTL system provides a simple, rapid and highly-sensitive detection of biological molecules with structural specificity. The QTL system provides a chemical moiety formed of a quencher (Q), a tethering element (T), and a ligand (L). The system is able to detect target biological agents in a sample by observing fluorescent changes.

The QTL system can rapidly and accurately detect and quantify target biological molecules in a sample. Suitable examples of ligands that can be used in the polymer-QTL approach include chemical ligands, hormones, antibodies, antibody fragments, oligonucleotides, antigens, polypeptides, glycolipids, proteins, protein fragments, enzymes, peptide nucleic acids and polysaccharides. Examples of quenchers for use in the QTL molecule include methyl viologen, quinones, metal complexes, fluorescent dyes, and electron accepting, electron donating and energy accepting moieties. The tethering element can be, for example, a single bond, a single divalent atom, a divalent chemical moiety, and a multivalent chemical moiety. However, these examples of the ligands, tethering elements, and quenchers that form the QTL molecule are not to be construed as limiting, as other suitable examples may be used.

Traditional PCR employs reiterative thermal cycling to amplify DNA. However, according to exemplary embodiments of the present invention DNA may be amplified without thermal cycling (e.g., by isothermal amplification). For example, DNA may be isothermally amplified by Loop-mediated isothermal amplification (LAMP), Strand displacement amplification (SDA), Nicking enzyme amplification reaction (NEAR), Recombinase Polymerase Amplification (RPA), Helicase-dependent amplification (HDA), or thermophilic helicase dependent amplification (tHDA). For instance, isothermal amplification is described in Oriero, E. C., et al. “Comparison of two isothermal amplification methods: Thermophilic helicase dependent amplification (tHDA) and loop mediated isothermal amplification (LAMP) for detection of Plasmodium falciparum” International Journal of Infectious Diseases 21 (2014): 381; and Li, Ying; et al. “Detection and Species Identification of Malaria Parasites by Isothermal tHDA Amplification Directly from Human Blood without Sample Preparation” The Journal of Molecular Diagnostics 15.5 (2013): 634-641.

According to exemplary embodiments of the present invention, DNA may be amplified by Loop-mediated isothermal amplification (LAMP). LAMP is also referred to as a single tube technique for amplifying DNA wherein all reagents are incubated in a single sample tube. LAMP utilizes a DNA polymerase with strand displacement properties, and a thermocycler need not be used. LAMP may employ a set of primers (e.g., 4 or 6 primers) targeting a set of regions (e.g., 6 or 8 regions) within a relatively small target DNA sequence. LAMP may employ 2 inner and 2 outer primers, plus 2 additional loop-primers, which may anneal at the loop structure in LAMP amplicons. This assay design enhances amplification sensitivity while accelerating reaction time. For instance, LAMP is described in Oriero, E. C., et al. “Comparison of two isothermal amplification methods: Thermophilic helicase dependent amplification (tHDA) and loop mediated isothermal amplification (LAMP) for detection of Plasmodium falciparum” International Journal of Infectious Diseases 21 (2014): 381; and Notomi. Tsugunori, et al. “Loop-mediated isothermal amplification of DNA” Nucleic acids research 28.12 (2000): e63-e63.

According to an exemplary embodiment of the present invention, DNA may be amplified by Strand Displacement Amplification (SDA). SDA is a non-sequence-specific method for DNA amplification. In SDA, random hexamer primers are annealed to a DNA template strand and DNA synthesis is performed by a high fidelity DNA polymerase. For instance, SDS is described in U.S. Pat. No. 5,455,166; U.S. Pat. No. 5,712,124; Asiello, Peter J., and Antje J. Baeumner. “Miniaturized isothermal nucleic acid amplification, a review” Lab on a Chip 11.8 (2011): 1420-1430; and Walker, G. Terrance, et al. “Strand displacement amplification—an isothermal, in vitro DNA amplification technique” Nucleic Acids Research 20.7 (1992): 1691-1696.

According to an exemplary embodiment of the present invention, DNA may be amplified by Nicking Enzyme Amplification Reaction (NEAR) amplification. NEAR amplification includes using a strand-displacing DNA polymerase to synthesize DNA from a nick created by a nicking enzyme. NEAR may produce many short nucleic acids from a target sequence relatively quickly. Alternating cycles of nicking and DNA extension may result in billion-fold amplification in about 5-10 minutes. For instance, NEAR is described in Ménová et al. “Scope and Limitations of the Nicking Enzyme Amplification Reaction for the Synthesis of Base-Modified Oligonucleotides and Primers for PCR” Bioconjugate Chemistry 24.6 (2013): 1081-1093.

According to an exemplary embodiment of the present invention, DNA may be amplified by Recombinase Polymerase Amplification (RPA). RPA is also referred to as a single tube technique for amplifying DNA. RPA my include three enzymes—a recombinase, a single-stranded DNA-binding protein (SSB) and strand-displacing polymerase. The recombinase may pair oligonucleotide primers with homologous sequence in duplex DNA. The SSB may bind to displaced strands of DNA and may prevent displacement of the primers. The strand displacing polymerase may start DNA synthesis at a point where the primer has bound to a target DNA. A reverse transcriptase enzyme may be added to an RPA reaction to detect RNA and DNA without the production of cDNA. For instance, RPA is described in Lutz et al. “Microfluidic lab-on-a-foil for nucleic acid analysis based on isothermal recombinase polymerase amplification (RPA)” Lab on a Chip 10.7 (2010): 887-893.

According to an exemplary embodiment of the present invention, DNA may be amplified by thermophilic helicase dependent amplification (tHDA). tHDA may selectively amplify a target DNA sequence (e.g., a relatively short DNA sequence of about 70 bp to 120 bp) defined by two primers. tHDA employs a helicase to separate DNA, instead of heat, to generate single stranded DNA templates for primer binding and extension by DNA polymerase. A single copy of DNA can be amplified by tHDA for detection. For instance, tHDA is described in Oriero, E. C., et al. “Comparison of two isothermal amplification methods: Thermophilic helicase dependent amplification (tHDA) and loop mediated isothermal amplification (LAMP) for detection of Plasmodium falciparum” International Journal of Infectious Diseases 21 (2014): 381; and Li, Ying, et al. “Detection and Species Identification of Malaria Parasites by Isothermal tHDA Amplification Directly from Human Blood without Sample Preparation” The Journal of Molecular Diagnostics 15.5 (2013): 634-641.

### Next-Generation Sequencing

According to exemplary embodiments of the present invention, DNA may be sequenced and/or detected by a next-generating sequencing (NGS) technology. NGS refers to a group of high-throughput sequencing technologies (e.g., massively parallel sequencing). NGS may sequence relatively large stretches of nucleic acid sequences or an entire genome. In NGS, many relatively small nucleic acid sequences may be sequenced simultaneously from a DNA sample and a library of small segments (i.e., reads) may be formed. The reads may then be reassembled to identify a large nucleic acid sequence or a complete nucleic acid sequence. For instance, as many as 500,000 sequencing operations may be run in parallel. For instance, MALBAC followed by traditional PCR may be used in NGS technologies. For instance, NGS is described in Mardis “The impact of next-generation sequencing technology on genetics” Trends in genetics 24.3 (2008): 133-141; and Metzker “Sequencing technologies—the next generation” Nature Reviews Genetics 11.1 (2009): 31-46.

Massively Parallel Signature Sequencing (MPSS) is an exemplary NGS technology. MPSS identifies mRNA transcripts by generating 17-20 base pair signature sequences. MPSS can be utilized to both identify and quantify mRNA transcripts in a sample. For instance, MPSS methods are described in Brenner, Sydney, et al. “Gene expression analysis by massively parallel signature sequencing (MPSS) on microbead arrays” Nature biotechnology 18.6 (2000): 630-634.

Polony Sequencing is an exemplary NGS technology in which millions of immobilized DNA sequences can be read in parallel. Polony sequencing has been found to be extremely accurate with a low error rate. Polony sequencing is a multiplex sequencing technique in which multiple analytes may be measured in a single run/cycle or a single assay. For instance, Polony Sequencing methods are described in Shendure et al. “Advanced sequencing technologies: methods and goals” Nature Reviews Genetics 5.5 (2004): 335-344; and Shendure et al. “Next-generation DNA sequencing” Nature biotechnol 26.10 (2008): 1135-1145.

Pyrosequencing is an exemplary NGS technology in which luciferase is utilized to detect individual nucleotides added to a nascent DNA. Pyrosequencing may amplify DNA contained in droplets of water in an oil solution. A droplet of water may include, for instance, one DNA template attached to a primer-coated bead. For instance, pyrosequencing methods are described in Vera, J. Cristobal, et al. “Rapid transcriptome characterization for a nonmodel organism using 454 pyrosequencing” Molecular ecology 17.7 (2008): 1636-1647; and Ronaghi “Pyrosequencing sheds light on DNA sequencing” Genome research 11.1 (2001): 3-11.

Illumina Sequencing is an exemplary NGS technology in which DNA molecules and primers are attached to a slide. The DNA molecules may be amplified by a polymerase and DNA colonies (i.e., DNA clusters) may be formed. For instance, lumina Sequencing methods are described in Hanlee “Next-generation DNA sequencing” Nature biotechnology 26.10 (2008): 1135-1145; and Meyer and Kircher. “Illumina sequencing library preparation for highly multiplexed target capture and sequencing” Cold Spring Harbor Protocols 2010.6 (2010): pdb-prot5448.

Sequencing by Oligonucleotide Ligation and Detection (SOLiD Sequencing) is an exemplary NGS technology in which thousands of small sequence reads (i.e., DNA fragments) are generated simultaneously. The sequence reads may be immobilized on a solid support for sequencing. SOLiD sequencing may generally be referred to as a sequencing by ligation method. For instance, SOLiD sequencing methods are described in Hanlee Ji. “Next-generation DNA sequencing” Nature biotechnology 26.10 (2008): 1135-1145; and Meyer, Matthias, and Ansorge, Wilhelm J. “Next-generation DNA sequencing techniques” New biotechnology 25.4 (2009): 195-203.

Ion Torrent Semiconductor Sequencing is an exemplary NGS technology in which released hydrogen ions are detected during DNA polymerization. Ion Torrent Semiconductor Sequencing is a sequence-by-synthesis method. A deoxyribonucleotide triphosphate (dNTP) may be introduced into a microwell containing a template DNA strand. When the dNTP is complementary to a leading template nucleotide, the dNTP may be incorporated into the complementary DNA strand and a hydrogen on may be released. For instance, Ion Torrent Semiconductor Sequencing methods are described in Quail, Michael A., et al. “A tale of three next generation sequencing platforms: comparison of Ion Torrent, Pacific Biosciences and Illumina MiSeq sequencers” BMC genomics 13.1 (2012): 341.

DNA Nanoball Sequencing is an exemplary NGS technology, in which small fragments of genomic DNA are amplified using rolling circle replication to form DNA nanoballs. Amplified DNA sequences can then be ligated through the use of fluorescent probes as guides. For instance, DNA Nanoball Sequencing methods are described in Ansorge, Wilhelm J. “Next-generation DNA sequencing techniques” New biotechnology 25.4 (2009): 195-203, and Drmanac et al. “Human genome sequencing using unchained base reads on self-assembling DNA nanoarrays” Science 327.5961 (2010): 78-81.

Heliscope Single Molecule Sequencing is an exemplary NGS technology that does not require ligation or FOR amplification. Heliscope Single Molecule Sequencing is a direct-sequencing method in which DNA may be sheared, tailed with a poly-A tail and then hybridized to the surface of a flow cell. Large numbers of molecules (e.g., billions of molecules) may be then sequenced in parallel. For instance, Heliscope Single Molecule Sequencing methods are described in Pushkarev et al. “Single-molecule sequencing of an individual human genome” Nature biotechnology 27.9 (2009): 847-850.

Single Molecule Real Time (SMRT) Sequencing is an exemplary NGS technology, in which DNA may be synthesized in relatively small well-like containers referred to as zero-mode wave-guides (ZMWs). Unmodified polymerases may be attached to bottoms of the ZMWs. The unmodified polymerases may be used to sequence the DNA along with fluorescently labeled nucleotides which flow freely in the solution. Fluorescent labels may be released from the nucleotides as the nucleotide is incorporated into the DNA strand. SMRT sequencing is an example of a sequencing-by-synthesis method. For instance, SMRT Sequencing methods are described in Flusberg et al. “Direct detection of DNA methylation during single-molecule, real-time sequencing” Nature methods 7.6 (2010): 461-465.

In an exemplary embodiment, the invention provides a system for in-field detection and authentication of an item marked with one or more markers selected from the group consisting of a DNA molecule, a protein, a peptide an optical reporter, a marker chemical and a digital code. The system may include a receptacle adapted for receiving a sample from an marked item of interest; one or more analyzer modules each adapted to detect presence of at least one of the markers using a specific detection method tailored to the marker, providing detection data, and an analyzer for receiving detection data from the one or more analyzer modules and determining presence or absence of the one or more markers in the sample; wherein the analyzer is configured to determine the authenticity of the marked item by confirming the presence of the one or more markers in the sample.

### Marker Detection

Examples of useful detection systems according to exemplary embodiments of the present invention include: PCR style DNA detection (digital PCR, Pyrosequencing, DNAe), Electrophoresis (capillary or gel electrophoresis microfluidics), nanodevices using protein pores for DNA sequencing, and microarray systems. Exemplary embodiments of the present invention include DNA and RNA detection using handheld, multi-test, or instrument-free USB devices. Sample collection, chip size, assay and readout style can be tailored to the product and user requirements. Exemplary embodiments of the present invention can be carried out by systems on chips at the world's largest foundries using the standard CMOS process that powers devices such as laptops and smartphones. This CMOS technology provides the ability to create chips with arrays of tens to millions of sensors, at ever decreasing cost, so that these chips essentially provide a laboratory on a small, single chip, which can be applied for solutions to an ever expending set of applications.

Ermantraut et al. (US2008/0207461 A1) disclose devices for conducting and analyzing microarray detection and discloses devices for the simultaneous performance of microarray experiments for quantitatively or qualitatively detecting specific interactions between probes and target molecules in a microtiter plate, as well as methods for manufacturing such devices. Such devices can duplicate and characterize nucleic acids simultaneously, as disclosed in Ehricht et al. (U.S. Pat. No. 7,888,074 B2). Devices have been built for detecting fluorescent signals (Ermantraut et al. US2004/0196455 A1), along with assay devices and methods for the detection of analytes (Ermantraut et al. US2011/0071038 A1), which can then be incorporated into a device for holding and detecting libraries of such substances (Ulrich et al. US2006/0147996 A1). Such microarray methods and devices are suitable for use in the methods and systems of the present invention for the detection and authentication of nucleic acids and other chemical markers.

In exemplary embodiments, the microarray device may be disposed in a reaction vial configured to receive a provided sample. The microarray device may include a custom probe microarray configured to automatically detect the presence of one or more target nucleic acid sequences in a provided sample. Custom probe microarrays may be designed to detect the presence of unique combinations of one or more nucleic acid sequences making up the distinctive marker that is unique to a particular article of interest. Custom probe microarrays may include oligonucleotide probes spotted onto a number of locations on the microarray. The oligonucleotide probes may each be complementary to a target oligonucleotide sequence of a particular distinctive marker. The oligonucleotide probes may hybridize with the complementary target oligonucleotide sequences to detect the presence of the target nucleic acid sequences in the provided sample. The custom probe microarray may include any suitable microarray, without limitation. The custom probe microarray may have any suitable dimensions, and may include one or more control spots or reference spots, as desired.

The reaction vial including the microarray device may be configured to record and/or transmit detection data to an integrated or external device for generating a distinctive marker detection report. For example, the reaction vial may automatically determine whether each nucleic acid sequence of a particular distinctive marker is present and transmit that data to an integrated or external device to generate a report indicating whether the provided sample includes the distinctive marker. If the presence of a distinctive marker is detected, a report may be generated indicating that the provided sample is from an authentic article. If the presence of the distinctive marker is not detected, a report may be generated indicating that the provided sample is not from an authentic article (e.g., a forged article). Microarrays to detect peptides or proteins may also be designed and implemented, without limitation.

In one embodiment, the microarray device may be configured to provide a signal in each well of the array or matrix in which a desired DNA is detected. The wells yielding a positive detection signal can be arranged in a particular configuration unique to a particular DNA marker. The configuration can be random or can be arranged in a particular design such as a logo or trademark, or to spell out a message. In an exemplary embodiment, the configuration can be recognized optically and the data may be transmitted to a server or stored locally. Up-converting phosphor reporters for nucleic acid microarrays is described in van de Rijke et al. “Up-converting phosphor reporters for nucleic acid microarrays” Nature biotechnology 19.3 (2001): 273-276.

According to an exemplary embodiment of the present invention, a determination of whether or not an article of interest is marked at all may be performed by a qualitative isothermal system that can detect the presence of a nucleic acid taggant on an article. For example, the determination of whether an article is marked or not may be formed by an Alere-i™ (i-NAT) apparatus, which is commercially available (Alere Technologies, Jena, Germany).

According to an exemplary embodiment of the present invention, a determination of whether or not the article of interest is marked with the distinctive marker may be performed by using an in-field detection instrument employing RT-PCR with sample in-answer-out analysis capability. For example, an Alere™ q point of care diagnostic testing platform may be adapted to detect the presence of the distinctive marker in a sample. The Alere™ q point of care diagnostic testing platform is commercially available (Alere Technologies, Jena, Germany). The Alere™ q point of care diagnostic testing platform is a fully automated nucleic acid testing platform that can automatically perform nucleic acid extraction, isolation, amplification and detection. Data analysis, data reporting and data storage can be completely automated.

According to an exemplary embodiment, a Later-flow strip test may be employed to determine the presence of a marker. Lateral-flow strip tests are designed to detect the presence or absence of a target analyte (e.g., nucleic acid) in a sample. Lateral flow tests are also referred to as lateral flow immunochromatographic assays. For instance, lateral flow tests are described in Corstjens et al. “Use of up-converting phosphor reporters in lateral-flow assays to detect specific nucleic acid sequences: a rapid, sensitive DNA test to identify human papillomavirus type 16 infection” Clinical chemistry 47.10 (2001): 1885-1893; and Posthuma-Trumpie et al. “Lateral flow (immuno) assay: its strengths, weaknesses, opportunities and threats. A literature survey” Analytical and bioanalytical chemistry 393.2 (2009): 569-582.

According to an exemplary embodiment of the present invention, a southern dot blot may be employed to determine the presence of a marker. Southern dot blot may be used to detect the presence of a biomolecule (e.g., protein or DNA). A mixture including the biomolecule of interest may be applied to a membrane without separating the biomolecule from the mixture and the biomolecule may be attached to the membrane as a dot. A nucleotide probe can then be applied to the biomolecule of interest to determine the presence or absence of the biomolecule. For instance, southern dot blots are described in Dubeau et al. “Southern blot analysis of DNA extracted from formalin-fixed pathology specimens” Cancer research 46.6 (1986): 2964-2969; and Englerblum et al. “Reduction of Background Problems in Nonradioactive Northern and Southern Blot Analyses Enables Higher Sensitivity Than ³²P-Based Hybridizations” Analytical biochemistry 210.2 (1993): 235-244.

In an exemplary embodiment of the detection method of the present invention, pH-mediated semiconductor ISFET technology is used in DNA detection which enables non-optical, scalable solutions for multiple applications such as, for example, rapid sample-to-answer solutions for genotyping single nucleotide polymorphisms (SNPs) to whole genome sequencing. The release of ions can be followed in real-time as they are released in a biological process.

In one embodiment of the methods and systems of the present invention, a silicon transistor is used to detect DNA and RNA. For example, when complementary nucleotides are incorporated into a growing chain during DNA synthesis, hydrogen ions are released. These can be detected as an electrical signal. Conversely, if there is no match, no hydrogen ions are released and no signal is detected. This principle can be used for all kinds of DNA and RNA analysis without the need for fluorescent dyes or labels or precision optics. The pH-mediated semiconductor (ISFET) technology uses unmodified reagents and unmodified microchip technology (CMOS). This provides the advantages that these systems are versatile, disposable, chip-based platforms that are fast, simple and scalable. In one embodiment sensors, circuits and active elements can be integrated onto, for example, a single CMOS (complementary metal-oxide-semiconductor, or non-volatile BIOS: Basic Input/Output System memory) chip, thereby providing a multifunction chip.

In exemplary embodiments, the quantity or concentration of the nucleic acid taggant within a collected sample can be determined and compared to the initial amount of nucleic acid taggant placed in the product to allow for the detection of fraud caused by diluting the product with inferior products by forgers. In general, quantitative detection methods comprise providing an internal or external control to evaluate the efficiency of detection from one sample/analysis to the next. The efficiency of detection may be affected by many parameters such as probe hybridization conditions, molecules or substances in the product which may interfere with detection, and/or primer integrity, enzyme quality, temperature variations for detection methods utilizing PCR. By providing a control in the detection methods variable conditions can be normalized to obtain an accurate final concentration of the nucleic acid tag in the product.

In an exemplary embodiment, the invention provides a method for in-field detection and authentication of a DNA marker, the method including providing a sample from an item of interest; analyzing the sample to detect the presence of a DNA marker encoding information unique to said item using a pore protein capable of forming a pore through which the DNA can pass base by base, and wherein the DNA sequence is detected through a characteristic change in an electrical field applied across the pore caused by the DNA passing through the pore; and the sequence data may be recorded and/or exported to a data storage device; and thereby verifying the authenticity of the item by determining that the DNA marker is present in the analyzed sample. DNA may pass through the pore a single base at a time, and thus characteristic changes to the electric field applied across the pore may be detected for each individual base.

In an exemplary embodiment, the invention provides a method for in-field detection and authentication of a DNA marker, including providing a sample from an item of interest; analyzing the sample to detect the presence of a DNA marker encoding information unique to said item using a sequence-specific DNA-binding moiety; and verifying the authenticity of the item by determining that the DNA marker is present in the analyzed sample. The sequence-specific DNA-binding moiety can be any suitable sequence-specific DNA-binding moiety, such as a sequence-specific DNA binding protein, a pore protein or an antibody.

The resultant data generated by such systems using the above methods can be encrypted and/or password protected. Analysis of the resultant data generated from such systems can be analyzed in a computer network system such as that disclosed by Grinkemeyer and Dailey (US 2011/001833 A1).

In an exemplary embodiment, the invention provides a method for in-field detection and authentication of a DNA marker, the method includes: providing a sample from an item of interest; analyzing the sample to detect the presence of a DNA marker encoding information unique to said item using a DNA sequencing method, wherein the DNA sequencing method is performed using a portable DNA sequencing device; and verifying the authenticity of the item by determining that the DNA marker is present in the analyzed sample. The DNA sequencing can be by any suitable method; such as for instance a method selected from Maxam & Gilbert chemical sequencing, dideoxy sequencing, Sanger sequencing, nucleic acid hybridization, nanopore sequencing, pyrosequencing or next generation sequencing, including ion-dependent detection or pH sensing. In exemplary embodiments, the DNA sequencing is performed using microarray hybridization. In an exemplary embodiment, the DNA sequencing is performed using dot blot hybridization.

A PCR sequence detection system may include an analysis device that is encrypted and/or password protected against unauthorized use. The analysis device can be linked to a server by a wireless connection or a hard wired connection. The server can be a stand-alone server or a network server; the network server can be any suitable network server, such as for instance a hardwired server or a cloud-based server. In one embodiment, the data verifying the authenticity is processed and stored on the alone server or the network server. In an exemplary embodiment, the data from the step of verifying the authenticity is processed and stored on the stand alone analysis device. In exemplary embodiments, the analysis device is modularized.

Tomazou et al. (U.S. Pat. No. 7,888,015 B2) employs QPCR using solid-state sensing, a pH sensor comprising an ion-sensitive field effect transistor (ISFET) to perform real time detection/quantification of nucleic acid amplification, based on detection of protons released during the primer extension phase. Tomazou et al. (U.S. Pat. No. 7,686,929 B2), utilizes this ISFET type of sensing apparatus to generate an electrical output signal in response to localized fluctuations in ionic charge or adjacent the surface of the transistor, and a means for detecting the electric output signal from the ISFET, the localized fluctuations of ionic charge indicating events occurring during a chemical reaction. The combination is summarized in Tomazou et. al. (US 2010/0159461 A1) which discloses a device for detecting chemical reactions, hybridization of nucleic acid sequences, and genetic variants, utilizing label free nucleic acid detection and interpretation. These processes and systems for hybridization, and sequencing of nucleic acid sequences or PCR amplicons derived from nucleic acid marker sequences can be configured for use in the methods and systems of the present invention for detection and authentication of nucleic acids and other chemical markers. A direct detection method for PCR-amplified DNA is described in Nazarenko et al. “A closed tube format for amplification and detection of DNA based on energy transfer” Nucleic Acids Research 25.12 (1997): 2516-2521.

In one embodiment, DNA may be bound to a fluorescent probe by Fluorescent in situ Hybridization (FISH). In FISH, a fluorescent probe may be bound to a portion of a fixed nucleic acid sequence complementary to that of the fluorescent probe. A target nucleic acid sequence can be bound at a desired position and the fluorescent probe may be viewed using fluorescence microscopy. For instance, FISH methods are described in Bianchessi et al. “Point-of-care systems for rapid DNA quantification in oncology” Tumori 94(2) (2008 March-April): 216-25. A site-specific functionalization of oligonucleotides is described in Agrawal et al. “Site-specific functionalization of oligodeoxy-nucleotides for non-radioactive labelling” U.S. Pat. No. 5,321,131.

In one embodiment, DNA detection may be performed using a BESt™ Cassette (Biohelix Corporation, Beverly, Mass.). BESt™ cassette detection is described, for instance, in Tang et al. “Nucleic acid assay system for tier II laboratories and moderately complex clinics to detect HIV in low-resource settings” Journal of Infectious Diseases 201. Supplement 1 (2010): S46-S51.

In one embodiment, the invention provides a method for in-field detection, wherein the distinctive marker and the specific detection method tailored to the distinctive marker pair is selected from the following marker and detection system pairs: a nucleic acid marker and a PCR detection system or hybridization detection system or a nucleic acid sequencing system; a protein marker and a HPLC chromatographic detection system or ELISA detection system, an optical reporter and a light detection system, and a chemical marker and a chromatographic detection system. In one embodiment, one of the detection device and the analysis device is modularized. In an exemplary embodiment, the detection device and the analysis device are each modularized.

In an exemplary embodiment, the invention provides a detection system for DNA detection and authentication, the method including a portable detector configured to detect the presence of a DNA marker encoding unique information of an item of interest from a sample of the item; and an analysis device operatively connected to the portable detector and configured to verify the authenticity of the item by determining whether the DNA marker is present in the sample analyzed by the portable detector. The portable systems of the invention for DNA detection are useful, for example, for anti-counterfeiting, brand protection, theft/crime prevention, provenance determination, authentication, tracking and tracing, as well as genotyping.

In one embodiment, the invention provides a method for in-field detection and authentication of a DNA marker, wherein the method includes providing a sample from an item of interest; analyzing the sample to detect for the presence of the DNA marker encoding information unique to said item using a polymerase chain reaction (PCR) method, wherein the PCR method may be performed using a portable thermocycler device; and verifying the authenticity of the item by determining that the DNA marker is present in the analyzed sample and wherein the verifying the authenticity is performed using an analysis device operatively connected to the PCR device. Verifying the authenticity of the item may be performed using any suitable method. In one embodiment, the detection and authentication of the DNA marker is performed by determining whether the DNA marker is present in the sample analyzed in a portable detector by detecting the presence of amplicons, i.e. amplified DNA copies of part of the DNA marker sequence amplified by a PCR method. The PCR method can be any suitable PCR method, such as for instance, of real time PCR, digital PCR or qPCR. Amplicons can be resolved and detected by any of a variety of standard well known methods, including electrophoresis, such as by gel electrophoresis or capillary electrophoresis.

### Protein, Antibody and Carbohydrate Detection

According to exemplary embodiments of the present invention, in addition to nucleic acid markers, taggants comprising one or more proteins and/or one or more carbohydrates may be detected.

Taggants comprising one or more carbohydrates may be detected by a carbohydrate microarray. A carbohydrate microarray is described in more detail in Feizi et al. “Carbohydrate microarrays—a new set of technologies at the frontiers of glycomics” Current opinion in structural biology 13.5 (2003): 637-645.

Taggants comprising one or more proteins may be detected by enzyme-linked immunosorbant assay (ELISA). ELISA is a relatively rapid, high-throughput, quantitative immunoassay for the selective detection of target antigens. The underlying principle behind ELISA includes antibody mediated capture and detection of an antigen with a measurable substrate. For instance, ELISA detection of proteins is described in more detail in Jia et al. “Nano-ELISA for highly sensitive protein detection” Biosensors and Bioelectronics 24.9 (2009): 2836-2841.

According to an exemplary embodiment, a determination of whether or not the article of interest is marked with the distinctive marker including an antibody or a protein may be performed by using an in-field detection instrument with sample in-answer-out analysis capability. For example, an Alere Determine™ device, such as the Alere Determine™ HIV-1/2 Ag/Ab Combo may be adapted to detect the presence of the distinctive marker including an antibody or a protein in a sample. Alere Determine™ devices are commercially available. The Alere Determine™ HIV-1/2 Ag/Ab Combo detects both anti-HIV antibodies and HIV proteins is (Alere Technologies, Jena, Germany).

The Alere Determine™ HIV-1/2 Ag/Ab Combo is a point-of-care rapid testing system adapted to detect specific antibodies. The Alere Determine™ platform allows screening and support in the detection of HIV, tuberculosis, hepatitis B, and syphilis in minutes.

### Authenticity Verification

In an exemplary embodiment, the present invention provides a detection system for DNA detection and authentication, including: a portable detector configured to detect the presence of a DNA marker encoding unique information of an item of interest from a sample of the item; and an analysis device, which can be any suitable analysis device, such as a stand-alone device, for instance a desktop computer, a laptop computer, a smartphone or a tablet, operatively connected to the portable detector and configured to verify the authenticity of the item by determining whether the DNA marker is present in the sample analyzed by the portable detector.

In one embodiment, the analysis device is configured to verify the authenticity of the item by determining whether the DNA marker is present in the sample analyzed by the portable detector by comparing the sample analyzed to a known sequence for the DNA marker stored in a database accessible in the analysis device. In exemplary embodiments, the verifying of the authenticity step of the method of the invention may include comparing the DNA marker sequence analyzed in the sample to a sequence of the DNA marker stored in a database accessible to or via the analysis device.

In an exemplary embodiment, the analysis device is configured to verify the authenticity of the item by determining whether the DNA marker is present in the sample analyzed by the portable detector by recognizing a pattern of a micro-array pattern to determine that the DNA marker is present. Alternatively, the analysis device is configured to verify the authenticity of the item by determining whether the DNA marker is present in the sample analyzed by the portable detector by detecting the presence of amplified or hybridized DNA detected by a complementary nucleic acid sequence.

In one embodiment, verifying of the authenticity of the article of interest is performed using an analysis device operatively connected to the portable DNA sequencing device. The analysis device can be any suitable analysis device, such as for instance a desktop computer, a laptop computer, a smartphone, a tablet, or the like. In exemplary embodiments, the analysis device is encryption and password protected against unauthorized use.

In one embodiment, the verifying of authenticity includes comparing the DNA marker sequence analyzed in the sample to a known sequence of the DNA marker stored in a database accessible via the analysis device. The DNA sequence can be stored locally in a stand-alone server or a network server operatively connected to the analysis device by a wired or a wireless connection. In an exemplary embodiment, the analysis device can be modularized. The results of the verification of authenticity can be processed and stored on a local server or a network server. The sequence of the DNA marker can be any suitable unique sequence. In one embodiment, the sequence of the DNA marker is a sequence of from about 20 to about 1000 bases.

The known sequence of said DNA marker can be any unique sequence for uniquely identifying the item, such as, for instance, a sequence of from about 20 to about 1000 bases. The item marked with the DNA marker can also be marked with a detectable reporter. In one embodiment, the detectable reporter and the DNA marker are in the same location on the item, so that the detectable reporter can be used as a sample identifier for the position of the DNA marker. Alternatively, the detectable reporter and the DNA marker can be associated with each other, so that obtaining a sample of the detectable reporter can be used as a guide to obtain a sample of the DNA marker for authentication and validation. In an exemplary embodiment, the detectable reporter of the method is selected from a chemical reporter, a digital reporter or a peptide reporter. In an exemplary embodiment, the detectable reporter is a digital reporter is a barcode.

The sequence detection system can include an analysis device that is encrypted and/or password protected against unauthorized use. The analysis device can be linked to a server by a wireless connection or a hard wired connection. The server can be a stand-alone server or a network server. In one embodiment, the analysis device may be modularized. In one embodiment, the data verifying the authenticity is processed and stored on the stand-alone server or the network server.

In one embodiment, the marked item to be authenticated by the system of the invention is selected from a microchip, a label, a badge, a logo, a printed material, a textile or a commodity. In an exemplary embodiment, the item to be authenticated by the system is selected from the group consisting of cash, a currency note, a coin, a gem, an item of jewelry, a musical instrument, a passport, an antique, an item of furniture, artwork, a property deed, a stock certificate, or a bond certificate. The detection system of the invention can be configured to provide authentication data directly to the user via a display, or to a secure location, or headquarters to provide data for communication to a customer, or for storage in a database or printing a report. The authentication data can be read as a display or signal, or alarm.

The analysis device of the method for in-field detection and authentication of DNA can be any suitable analysis device, such as for instance a desktop computer, a laptop computer, a smartphone, or a tablet, etc. These devices may be operatively configured to communicate as a system in a network. The network can be any suitable network, such as for instance a network with one or more connections without limitation, by RF, Wi-Fi, Bluetooth or a hard-wired connection.

### In-Field Detection Instruments

The in-field detection instrument may include a microsystem with sample in-answer out analysis capability. The microsystem may be a self-contained unit that performs all necessary analysis processes without the need for additional lab equipment. The microsystem may by automated, and may only require the addition of the sample to the microsystem and activation of the microsystem to perform the analysis. The in-field detection instrument may be portable or fixed in a single location. Sample in-answer out analysis refers to the ability of the microsystem to perform all analysis steps after transferring the sample to the microsystem and automatically providing a result. The microsystem may be configured to provide detection with a minimum of necessity for monitoring or adjustment by the operator. In an exemplary embodiment, the sample is loaded directly or from a sample collection device configured to mate with a sample port of the microsystem. The microsystem is then activated and the in-field detection instrument provides detection data with operator interaction. Detection data may be stored and/or exported. In the case of detecting the presence of a distinctive marker, a sample suspected of including the distinctive marker may be provided and transferred to the microsystem of the in-field detection instrument, and the microsystem may automatically determine whether or not the distinctive marker is present in the sample. Exemplary microsystems for in-field detection are described in more detail below.

The in-field detection instrument may communicate with a server comprising authenticity data for the article of interest. The server may comprise an authenticity data database storing profile information for a number of distinctive markers associated with a number of articles of interest. Authenticity data may be a unique profile corresponding to the distinctive marker. For example, the distinctive marker may include one or more unique nucleic acid sequences, and the authenticity data may be a digital copy of the unique nucleic acid sequences. An example of a suitable remote authentication server including an authentication database is described in Zorab (WO 01/99063).

### Microfluidic Sample Preparation Devices

A system utilizing microfluidics which are suitable for use in exemplary embodiments of the methods of the present invention is disclosed in Jovanovich et al. (U.S. Pat. No. 7,745,207 B2). Further, nanofluidics as disclosed by Janovich et al. (US2012/0115189 A1) and sample handling as disclosed in Green (U.S. Pat. No. 8,110,397 B2) are useful in additional embodiments of the present invention. A PCR device as described in Green (U.S. Pat. No. 7,170,594 B2) can also be used in exemplary embodiments of the present invention. Analysis methods have been developed using microfluidic systems and fractionation or partitioning of DNA solutions down to about one DNA molecule per droplet to identify polymorphisms in a quantum dot (Lo et al., US2009/0263580 A1). Methods and devices for digital PCR including the use of “droplet-in-oil” technology useful in the practice of embodiments of the present invention are disclosed by Davies et al. (US2010/0092973 A1). Oligonucleotide sequences have been used for the detection of the ricin gene and the ricin toxin (Czajka, US 2006/0240447) and a kit was created to detect this specific DNA in a sample.

According to exemplary embodiments of the present invention, microfluidic devices may be used for extraction, purification and stretching of a DNA sample. For instance, a microfluidic device for extraction, purification and stretching of human DNA from single cells is described in Benitez et al. “Microfluidic extraction, stretching and analysis of human chromosomal DNA from single cells” Lab on a Chip 12.22 (2012): 4848-4854. A microfluidic DNA chip is described in Zhao et al. “Electrochemical DNA detection using Hoechst dyes in microfluidic chips” Current Applied Physics 12.6 (2012): 1493-1496. A direct ultra-fast PCR for forensic genotyping is described in Aboud, “The development of direct ultra-fast PCR for forensic genotyping using short channel microfluidic systems with enhanced sieving matrices” (Jan. 1, 2012). ProQuest ETD Collection for FIU. Paper AAI3541755.

For instance, microfluidic devices generally, and microfluidic capillary electrophoresis devices in particular, are described in Wu, Jinbo, et al. “Extraction, amplification and detection of DNA in microfluidic chip-based assays” Microchimica Acta (2013): 1-21; Liu et al. “Integrated DNA purification, PCR, sample cleanup, and capillary electrophoresis microchip for forensic human identification” Lab on a Chip 11.6 (2011): 1041-1048; U.S. Pat. No. 7,745,207 B2 to Jovanovich et al.; and published US Patent Application US2012/0115189 A1 to Jovanovich et al.

According to exemplary embodiments of the present invention, one or more samples may be provided including one or more target oligonucleotide sequences, and one or more non-target oligonucleotide sequences (e.g., camouflage or decoy sequences). The target oligonucleotides may be purified by separating the target oligonucleotides from the camouflage oligonucleotides. The sample may be washed over a bed of substrates immobilized in a series of microchannels. The substrates may include one or more oligonucleotide probes that are complementary to the target oligonucleotides and are therefore configured to capture the target oligonucleotides through hybridization. The substrate may be any suitable substrate. For example, the substrate may be a magnetic bead with oligonucleotides probes bound thereto. The target oligonucleotides may bind the oligonucleotide probes to form a oligonucleotide-probe conjugate. The substrates (e.g., magnetic beads) bound to the target oligonucleotides may be transferred to a detection module to determine whether each target oligonucleotide is present. The substrates (e.g., magnetic beads) bound to the target oligonucleotides may be transferred to an amplification device or an amplification chamber. Amplified products may be transferred, for example, to a capillary electrophoresis column. Oligonucleotide separation may be performed by the application of an electric field according to standard capillary electrophoresis techniques known in the art. A laser with an optical detector system may be employed to detect the presence of the target oligonucleotides. Capillary electrophoresis and DNA detection techniques are described in Schwartz, Guttman. Separation of DNA by capillary electrophoresis. Beckman, 1995.

### Integrated Microsystems

According to exemplary embodiments of the present invention, a nucleic acid marker may be detected by an integrated microsystem. Integrated microsystems may include, for example, a microfluidic chip, a fully integrated microdevice or a lab on a chip configuration. Integrated microsystems can prepare a sample, amplify a target nucleic acid sequence (if necessary) and detect the presence of one or more target nucleic acid sequences on a single device. All steps can be performed on a self-contained, automated microsystem without the need for performing any manual testing in a lab. For instance, integrated microsystems are described in Wu et al. “Extraction, amplification and detection of DNA in microfluidic chip-based assays” Microchimica Acta (2013): 1-21; Liu et al. “Integrated DNA purification, FOR, sample cleanup, and capillary electrophoresis microchip for forensic human identification” Lab on a Chip 11.6 (2011): 1041-1048; and Ullrich, Thomas, et al. “Competitive Reporter Monitored Amplification (CMA)-Quantification of Molecular Targets by Real Time Monitoring of Competitive Reporter Hybridization” PloS one 7.4 (2012); e35438.

In other exemplary embodiments, the sample containing the nucleic acid taggant can be separated from other sample components, such as by capillary action in a microfluidic channel system of a power-free microchip, and detected by binding to a labelled immobilized hybridization probe which is complementary to at least a portion of the nucleic acid taggant sequence. The nucleic acid taggant can be detected directly or amplified (e.g. by isothermal amplification or by PCR) and detected by laminar flow assisted dendritic amplification (LFDA) using a detectable antibody specific for the label bound to the immobilized hybridization probe and detected with a second antibody in a dendritic cascade reaction, as is well known in the immunoassay art. See for instance: Hosokawa et al. DNA Detection on a Power-free Microchip with Laminar Flow-assisted Dendritic Amplification, Anal. Sci. (2010) 26: 1053-1057.

According to an exemplary embodiment of the present invention, an integrated microsystem may employ an encapsulated microarray system without automated fluorescent labeling and detection. The integrated microarray system can amplify one or more target sequences from a provided sample by using PCR or by isothermal amplification, fluorescently label one or more of the amplified products and detect the presence of the fluorescently labeled products. For instance, a microarray-in-a-tube detection system is described in more detail in Liu et al. “Microarray-in-a-tube for detection of multiple viruses” Clinical chemistry 53.2 (2007): 188-194.

According to an exemplary embodiment, a device for in-field detection of a distinctive marker includes an in-field detection instrument including a microsystem configured to perform sample in-answer out analysis. The in-field detection instrument is configured to analyze a sample to determine the presence of a distinctive marker in the sample. According to an exemplary embodiment, the in-field detection instrument may be configured to perform any suitable chemical or physical characterization method capable of determining one or more distinctive characteristics of the distinctive marker.

According to an exemplary embodiment, the sample collection unit may be configured to be directly coupled to the in-field detection device.

According to an exemplary embodiment, the integrated microsystem may employ a fluorescence background displacement technique. An array of immobilized oligonucleotide capture probes may be disposed in the reaction chamber having the reaction mixture. A solution comprising fluorescently labeled reporter oligonucleotide probes that are complementary to the immobilized oligonucleotide capture probes may also be provided in the reaction chamber. Amplicons generated in the reaction chamber that are complementary to the reporter oligonucleotide probes may compete with the capture probes for binding with the reporter oligonucleotide probes. That is, the reporter probes may each also be complementary to a specific target oligonucleotide sequence. Thus, as the number of copies of a particular amplicon (i.e., target oligonucleotide sequences) increases, the fluorescent signal detected in the corresponding immobilized oligonucleotide capture probe decreases because fewer reporter oligonucleotide probes bind the corresponding capture probe. This may allow simultaneous qualitative and quantitative detection of target nucleic acid sequences included in the oligonucleotide sequences of the sample. Optical images of the capture probe may be obtained at any desired time point, including at baseline before a sample has been added to the reaction mixture. Binding of non-target nucleic acid sequences (e.g., camouflage sequences) in the sample to the reporter probes may be avoided because of the hybridization specificity between the reporter probes and the target nucleic acid sequences. The capture probes may also be configured for direct binding between the target nucleic acid sequence and the capture probe. The reaction mixture holding unbound reporter probes may be displaced so that only bound reporter probes (bound to the capture probes) are detected. The reaction mixture may be displaced through any suitable displacement mechanism. For instance, mechanical fluorescence background displacement techniques are described in Ullrich et al. “Competitive Reporter Monitored Amplification (CMA)-Quantification of Molecular Targets by Real Time Monitoring of Competitive Reporter Hybridization” PloS one 7.4 (2012): e35438.

Exemplary integrated microsystems are described in more detail below.

### Informational DNA Coding

The enormous coding capacity of nucleic acid sequences is well known in the art. In exemplary embodiments of the present invention the nucleic acid markers used to identify an item and nucleic acid taggants used to encode information content of the tagged item may be detected in an array. The array may be any suitable array, such as a microarray comprising spots of capture DNAs, each spot including bound DNA molecules including a unique sequence complementary to the nucleic acid marker or one of the many possible nucleic acid taggants affixed or added to the item in or on labels, tags or packaging. The array is suitable for hybridization of the nucleic acid marker, nucleic acid taggants or amplicons derived therefrom. The hybridization array can be in any suitable format, such as for instance a plate, an array of microtiter wells, a matrix of spots, colonies, or a microarray.

The hybridization array may have suitable size and/or any arrangement of individual spots of DNA complementary to a nucleic acid marker or to a nucleic acid taggant. For example, the array may include a 1,000 by 1,000 grid of spots. In another example, the array may be arranged in a grid of 30,000 by 30,000 or more, so that 1 billon or more spots may be included in a single array. According to exemplary embodiments, the arrangement of sample spots may be employed to provide authenticity data for an article of interest, as described in more detail below.

In an exemplary embodiment, a plurality of “monoclonal colonies” of oligonucleotides may be attached to a solid surface to form a custom probe array, i.e. each spot or “monoclonal colony” includes multiple copies of a DNA sequence complementary to a nucleic acid marker or to a nucleic acid taggant. The monoclonal colonies may be submicrometer-sized colonies generated by PCR or by isothermal amplification on a solid surface, e.g. the surface of a next-generation sequencing microfluidic flowchip. The solid surface may have any desired size and the positions of the monoclonal colonies on the surface may be in any desired arrangement. For example, the monoclonal colonies may be arranged in a 1,000 by 1,000 colony grid. The solid surface may be any suitable surface, such as for instance, the surface of a microscope slide having a polyacrylamide gel coating. One example of the generation of immobilized monoclonal colonies of DNA templates is described in Ma, et al. “Isothermal amplification method for next-generation sequencing,” Proc. Natl Acad Sci 110.35 (2013): 14320-14323.

According to further exemplary embodiments, the custom probe array may include a group of randomly arranged oligonucleotide hybridization probes that are complementary to one or more nucleic acid marker sequences or to nucleic acid taggant oligonucleotide sequences. A plurality of camouflage oligonucleotide probes (e.g., decoy probes) may be disposed throughout the custom probe array at one or more positions of the array that are not occupied by the oligonucleotide hybridization probes to provide non-specific binding to reduce any background noise. The camouflage oligonucleotides are preferably not complementary to any of the target nucleic acid sequences. The custom probe array may include any desired number of oligonucleotide sequences not complementary to the nucleic acid marker or nucleic acid taggant oligonucleotide sequences. For example, the camouflage probes may include 1,000 to 1,000,000 or more different nucleic acid sequences which do not hybridize to any of the targeted nucleic acid marker or nucleic acid taggant oligonucleotide sequences. The nucleic acid marker may hybridize to the spots of complementary sequences of immobilized oligonucleotide hybridization probes, and a unique “fingerprint” may be generated. The unique fingerprint corresponds to a particular physical arrangement of the oligonucleotide hybridization probes on a particular array or chip. Thus, the custom probe array can provide a positive identification of an article of interest in at least two ways: first, by positively identifying the presence of a nucleic acid marker oligonucleotide sequence or of a nucleic acid taggant, and second, by positively identifying the presence of the unique fingerprint, wherein positively identifying the presence of the oligonucleotide sequence in the nucleic acid marker, or nucleic acid taggant does not require knowing the physical arrangement of the custom probe array corresponding to the unique fingerprint.

According to an exemplary embodiment, the bound oligonucleotide hybridization probes are not randomly arranged, but rather form a recognizable symbol or pattern. The oligonucleotide hybridization probes may be positioned at desired locations on the array to form a custom fingerprint. The custom fingerprint may include any desired number of oligonucleotide hybridization probes disposed at any combination of positions on the custom probe array. For example, the probes of the custom probe array may be arranged in a grid, and the positions of the oligonucleotide hybridization probes may be arranged as a “plus” symbol or a star shape positioned at a specific location within the array or grid. The positions of the probes on the custom probe array not occupied by the oligonucleotide hybridization probe specific to the article of interest may be occupied by other oligonucleotide hybridization probes so that the array may be useful for detection of several different nucleic acid markers, or nucleic acid taggants. Alternatively, the positions of the probes on the custom probe array not occupied by the oligonucleotide hybridization probes may be occupied by camouflage probes or other DNA sequences. The camouflage probes may include any desired number of oligonucleotide sequences. For example, the camouflage probes may include 1,000 different nucleic acid sequences which are randomly arranged, and which preferably do not hybridize to any of the targeted oligonucleotide sequences. In an exemplary embodiment, the custom probe array may include an arrangement oligonucleotide hybridization probes specific to the item of interest forming letters, words, or even letters and symbols or binary code.

More than one oligonucleotide may be employed in the nucleic acid marker for a particular item. The custom probe array may also include more than one oligonucleotide hybridization probe corresponding to the nucleic acid marker oligonucleotide or nucleic acid taggants affixed to the article of interest or its packaging to provide redundancy and extra security and confirmation of detection, authentication, tracking and validations.

The arrangement of the oligonucleotide hybridization probes may also be used to code for additional information about the article of interest beyond its authenticity. Additional nucleic acid taggants may be added or affixed to the article of interest at various stages of manufacturing or packaging and distribution, each corresponding to a different oligonucleotide hybridization probe in the array used for detection, validation or tracking of the item and may be used to represent or encode any desired information about the article of interest. For example, the presence or absence of hybridization to a specific oligonucleotide hybridization probe at a specific spot location in the array or the arrangement of multiple copies of the specific oligonucleotide hybridization probe may be used to encode specific information about the article. The encoded information can be any desired information, such as for instance and without limitation, the model, serial number, geographic origin, component parts used and date of manufacture, distributor and final seller or purchaser of the article of interest.

A method for labeling an object or product with a specified nucleic acid tag and then detecting the nucleic acid tag in the object or product in an effective manner is described.

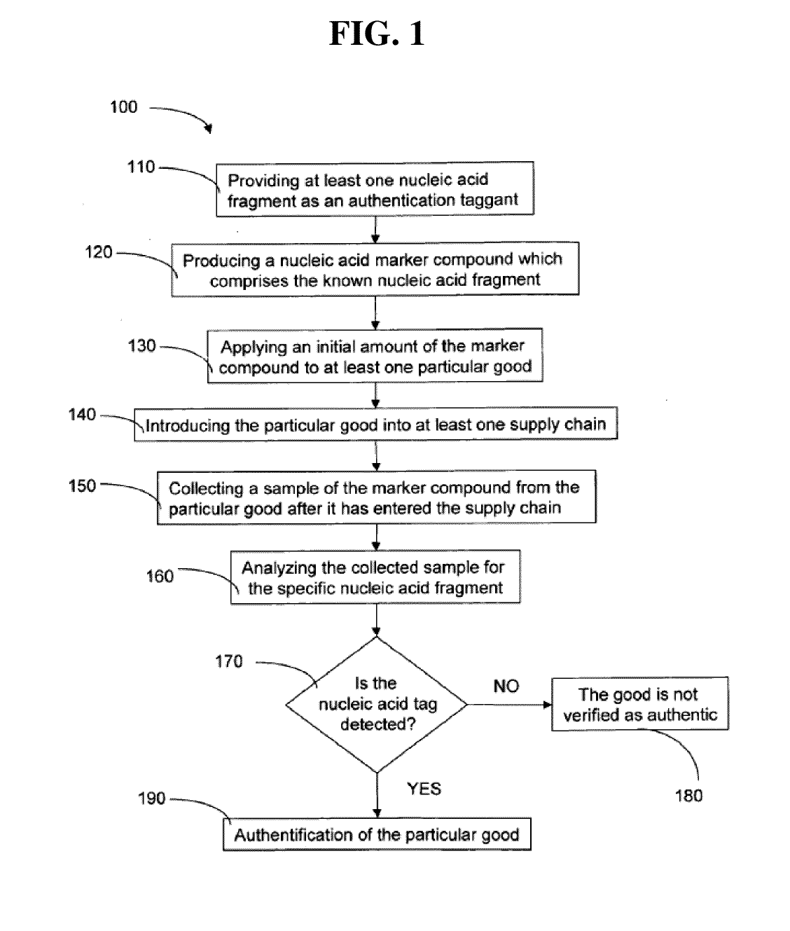

FIG. 1 shows a flow chart of the process 100 of introducing a nucleic acid tag into or onto a product and being able to detect the nucleic acid tag or marker incorporated in the product. Referring to FIG. 1, the process comprises providing at least one specific nucleic acid fragment as an authentication tag or marker for a product in event 110. In event 120, a nucleic acid marker compound which comprises a known nucleic acid fragment is produced. The nucleic acid marker may be DNA, cDNA, or any other nucleic acid fragment comprising nucleic acids or nucleic acid derivatives. The marker maybe a nucleic acid fragment that is single stranded, or double stranded and may vary in length, depending on the product to be labeled as well as the detection technique utilized in the nucleic acid marker detection process.

After the nucleic acid fragment with a known nucleic acid sequence has been manufactured or isolated, and added to a marker compound mixture, the process for authenticating a particular product by detecting a nucleic acid tag, further comprises applying a predetermined amount of nucleic acid marker to a specified product in event 130. The particular product may be tagged with a nucleic acid marker throughout the complete product or only in a predetermined region of the product. When the product to be authenticated is a solid, a specified amount of nucleic acid marker may be incorporated throughout the volume of the product, only on the surface of the product or in some exemplary embodiments, placed only on a previously designated section of the product. When the product is a prescription drug, either in solid or liquid form, the drug (e.g., pills, gel capsules, etc.) could have the nucleic acid tag incorporated completely throughout the product. If the product is a prescription drug in tablet form, the nucleic acid marker compound may be in the form of a solid which can be introduced into the product (drug) during the compression of the drug into a tablet. If the product is a textile garment, the marker could be either solid or liquid and applied to a predetermined area of the garment. Textiles may have a label with the manufactures name on it and may also be used as a region of the product which the nucleic acid marker is placed. The above examples are presented for clarity and are not meant to be limiting in scope.

The embodiment of the method of authenticating a product depicted in FIG. 1 further comprises introducing the marked product into a supply chain or placing the product into service in event 140. Frequently, forgers have the best access to products when they are being shipped from the manufacturer/producer to a retail outlet or location. Forgers also have access to the products of interest during maintenance or service of certain of products, such as aircraft, where the product of interest is inspected or replaced (e.g., fasteners). Having a method in which the producer can track and authenticate its products allows for a better monitoring of when and where products are being replaced with forgeries or being tampered with.

In event 150, a sample is collected from the particular product comprising the nucleotide tag after it has entered the supply chain or been in service. A manufacturer or an authorized individual can collect a sample of the marker from the product at any desired point along the supply chain or during the service or routine maintenance of an item where the product is utilized for authentication purposes. In exemplary embodiments, this may comprise visually inspecting the marker, and/or scrapping, cutting or dissolving a portion of the marker compound in a solvent for analysis.

The embodiment shown in FIG. 1 further comprises analyzing the collected sample for the presence of the nucleic acid taggant in event 160. The analysis of the sample collected from the product may occur without further purification, but some extraction, isolation or purification of the nucleic acid tag obtained in the sample may be required. Details on the extraction, concentration and purification techniques useful for the methods of the invention are described more fully below and also in the examples provided herein.

Analyzing the sample may include providing a “detection molecule” configured bind to the nucleic acid tag. A detection molecule includes, but is not limited to, a nucleic acid probe and/or primer set which is complementary to the sequence of the nucleic acid taggant, or a dye label or color producing molecule configured to bind and adhere to the nucleic acid taggant. When the detection of the nucleic acid taggant comprises amplifying the nucleic acid taggant (e.g., by using PCR), the detection molecule(s) may be primers which specifically bind to a certain sequence of the nucleic acid taggant. When real time PCR is utilized in the analysis of the sample, an identifiable nucleotide probe may also be provided to enhance the detection of the nucleic acid taggant, as well as to provide semi-quantitative or quantitative authentication results. With the use of real time PCR, for example, results from the analysis of the sample can be completed within 30 minutes to 2 hours, including extracting or purifying the nucleic acid taggant from the collected sample. Exemplary embodiments utilize a wide range of detection methods besides PCR and real time PCR, such as fluorescent probes, probes configured to molecules which allow for the detection of the nucleic acid tag when bound to the probe by Raman spectroscopy, Infrared spectroscopy or other spectroscopic techniques used by those skilled in the art of nucleic acid detection.

In event 170 the results of the analysis of the collected sample are reviewed to determine if the specific nucleic acid taggant was detected in the sample. If the nucleic acid taggant is not found or detected in the collected sample of the product of interest, the conclusion from the analysis is the that product is not authentic or has been tampered with at event 180 of FIG. 1. If the nucleic acid taggant is detected in the sample at event 190, then the product is verified as being authentic.

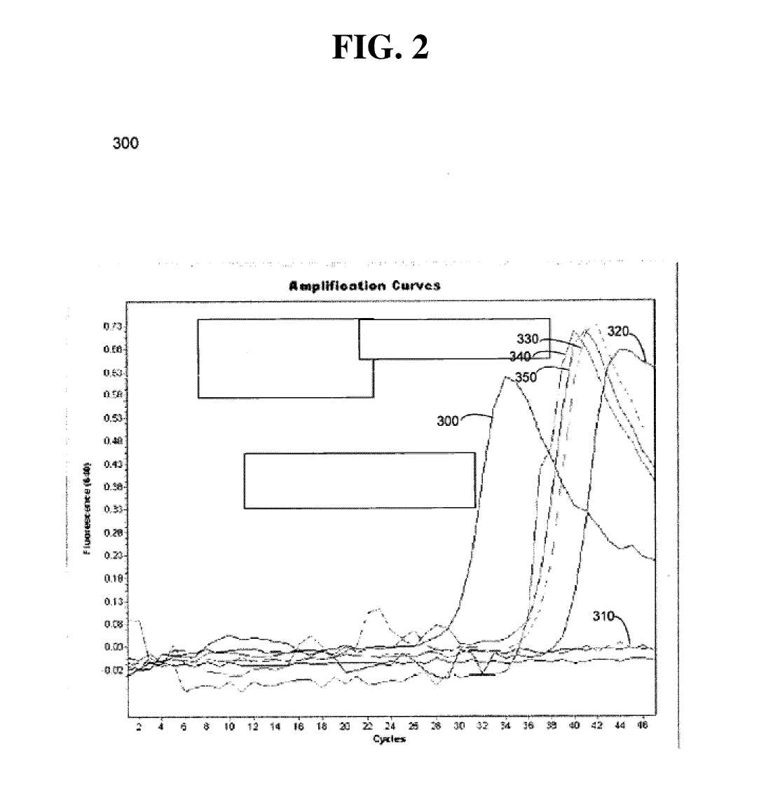

FIG. 2 is a graphical representation of real time PCR results illustrating the detection of a DNA taggant from the fastener in accordance with one embodiment of the methods of the invention. FIG. 2 shows real time PCR results of torque seal samples which were subjected to the following extraction methods. All of the samples A, B, D, and E were dried for 20 days and then subjected to grinding. Samples D and E were further treated by boiling the grinded sample in water. A positive control 300 for the DNA marker as well as replicate negative controls 310 were also subjected to the real time PCR conditions as the torque seal samples. The positive control 300 has a Cp value of 29 while the negative controls 310 gave only a background signal. The two torque seal samples which were only subjected to grinding, samples A 320 and B 330, gave Cp values of 37 and 33, respectively. The samples which were ground and boiled, samples D 340 and E 350, both had a Cp value of 34. These results demonstrate that the DNA marker can be detected from a sample of torque seal material after exposure for 20 days. Both methods gave qualitative results with the method of grinding followed by boiling giving semi-quantitative results.

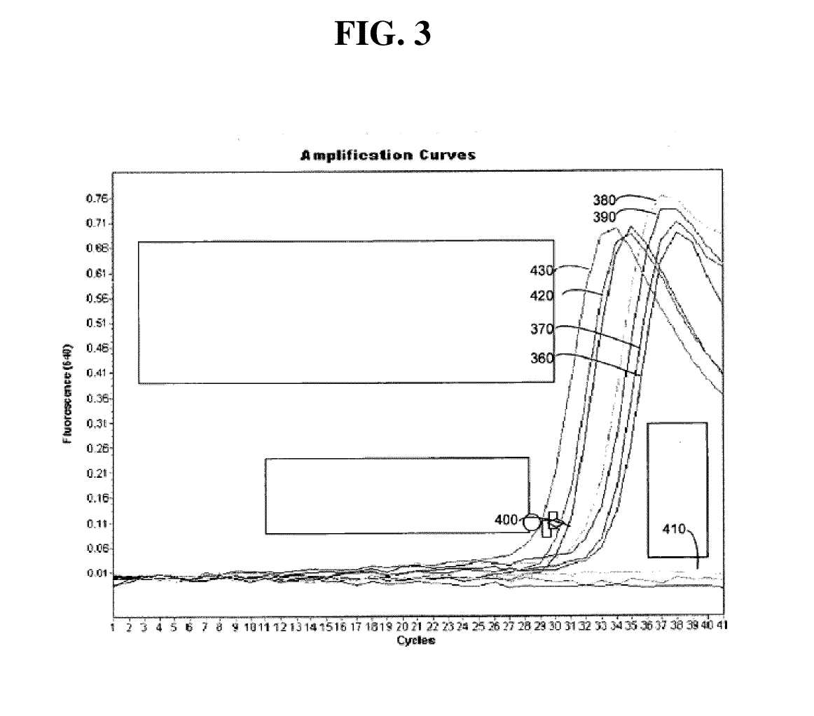

FIG. 3 shows real time PCR results of torque seal samples which were subjected to the following extraction methods. All of the samples were dried for 20 days, subjected to grinding, dissolved in water and further purified using a QIAgen PCR Purification Kit to remove particulate material that could interfere with the optics of the PCR instrument. The purification step involves centrifugation to bind the DNA present in the torque seal sample to a filter, then wash away any impurities, followed by eluting the DNA with water. The procedure is fast and easy, and takes no more than 15 minutes to perform. Following clean-up, real-time PCR was performed on samples A and B using 2 μL or 4 μL of purified extracted torque seal DNA. We also spiked the pooled samples A and B with 2 μL of positive control DNA (9543-2) to test for inhibitors in the PCR reaction.

The amplification curves of samples A and B after PCR clean-up are shown in FIG. 3. The 2 μL samples of A and B are curves 360 and 370 respectively, and gave similar Cp value of 32. The 4 μL samples of A and B, curves 380 and 390 gave a Cp about 30 and were also fairly reproducible. The positive control 400 amplified as expected with a Cp value of 29 while the replicate negative controls 410 gave no signal above background. Torque samples A and B were spiked with the positive control 420 and 430, respectively, to insure that the purification kit did not affect the amplification of the target DNA. The spiked A and B samples gave Cp values of 27 and 29 respectively. The fluorescent signal generated by samples subjected to the purification kit are smooth and have a typical shape curve for real time PCR. This experiment demonstrates the ability to detect a specific DNA marker from dried torque seal material. The results also indicate that the detection method can be semi-quantitative.

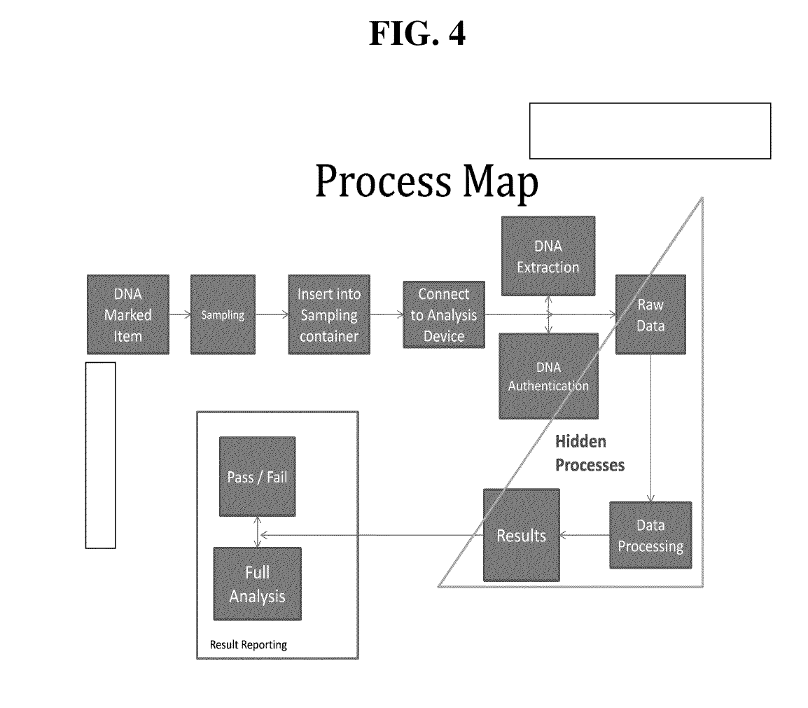

FIG. 4 shows an embodiment of a process according to the present invention. An item carrying a DNA marker is sampled, the sample is transferred to a sampling container which is operatively connected to an analysis device. The sample can be subjected to a DNA extraction process step, as appropriate for the particular sample and detection and authentication steps are carried out. Raw data is communicated to a data processing unit. Data from the processing unit can then be relayed to a local or remote processor or database for further analysis, display, storage or incorporation into a report, such as for instance, a “Pass/Fail” readout. Processes inside the triangular region and the rectangular analysis and readout regions are hidden, secure processes.

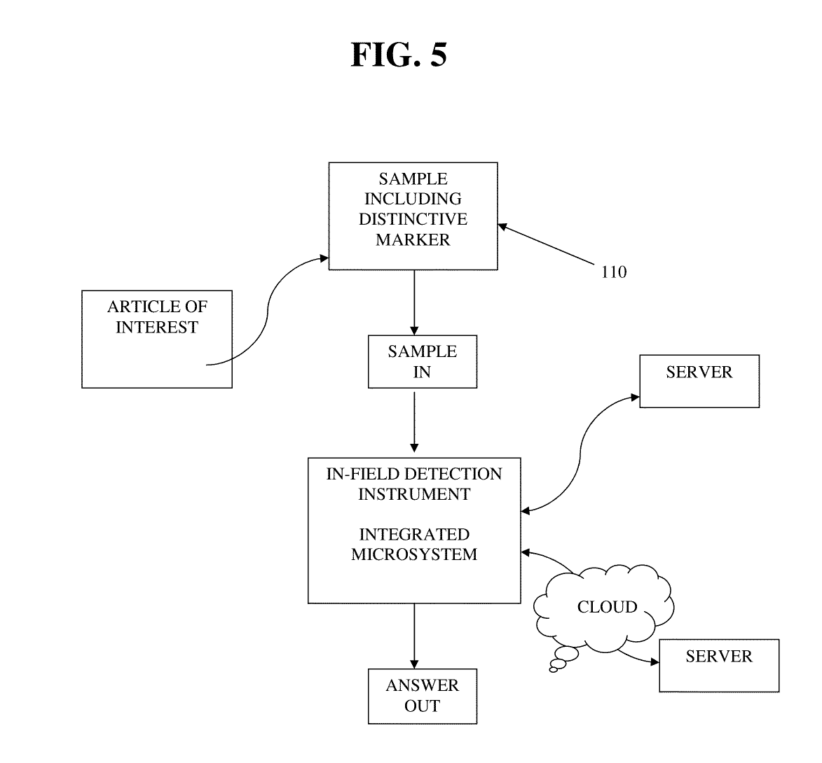

FIG. 5 shows an in-field detection instrument according to an exemplary embodiment of the present invention. A sample 110 is collected from an article of interest. The sample 110 is transferred to the in-field detection instrument. The in-field detection instrument includes an integrated microsystem with sample in-answer out analysis capability to detect the presence of the distinctive marker. The in-field detection instrument may provide a direct answer-out, for example as a readout on a screen or printout. Alternatively, or in addition, the in-field detection instrument may communicate with a server to compare data generated by the in-field detection instrument with data stored in the server. The server may be a free standing server or the server may be in the cloud, and the server in the cloud may in turn be linked to a free standing server.

### Example 1

### An Integrated Microsystem and Method for Rapid in-Field Detection of a Distinctive Marker

According to an exemplary embodiment of the present invention, the location of a distinctive marker including one or more nucleic acid sequences is identified on an article of interest. A sample of the distinctive marker including the nucleic acid sequences is obtained, and the sample is transferred to an array tube including a custom array chip. The sample is selectively fluorescently labeled and the presence of one or more nucleic acid sequences is determined by microarray analysis.

An array tube (AT) with a custom probe microarray in a microreaction vial may be employed. The array tube may be, for example, an Alere Technologies GmbH ArrayTube with a custom probe microarray (i.e., biochip) integrated into a microreaction vial, which is commercially available (Alere Technologies, Jena, Germany). The custom array includes oligonucleotides that are complementary to nucleic acid sequences making up the distinctive marker that is unique to the article of interest. The custom array chip includes a custom selection of complementary oligonucleotides probes that are spotted onto the array chip. The oligonucleotides probes hybridize with complementary target nucleic acid sequences to detect the presence of the target nucleic acid sequence in a sample.

The probe microarrays are customized for individual applications with oligonucleotide, polynucleotide or protein/peptide probe molecules. Each AT chip with dimensions of 3 mm×3 mm can be provided with up to 14×14 features including reaction control and reference marker spots.

Depending on the individual assay, nucleic acid as well as protein and peptide based arrays can be manufactured. Each AT is labeled with a unique data matrix code (a two-dimensional bar code) identifying array and related assay. AT testing is performed in combination with the method of precipitation staining, which is suited to both serological and nucleic acid based analysis. Catalytically induced precipitation directly correlates to the amount of target molecules binding to the array. Analysis of the resulting precipitation pattern is done by simple transmission measurements, leading to the effective reduction of the input in optical equipment. Transmission measurement may be taken with an ATR 03 instrument, which is commercially available (Alere Technologies, Jena, Germany) or an ARRAYMATE instrument, which is also commercially available (Alere Technologies, Jena, Germany).

Data is automatically recorded and analyzed directly from the array tube comprising the custom array chip and results are electronically communicated to a desired computer server or stored to a local disk. Data captured from the automated microarray may be automatically processed and compared with a database of known nucleic acid sequence combinations for authentication.

### Example 2

### An Integrated Microsystem and Method for Rapid in-Field Detection of a Distinctive Marker

A sample of a DNA marker of interest is provided and transferred to the integrated microsystem device for analysis. The sample includes one or more oligonucleotide sequences. The oligonucleotide sequences may include one or more target oligonucleotide sequences of the distinctive marker and one or more non-target oligonucleotide sequences as camouflage or carrier decoy sequences.

The sample includes a number of oligonucleotide sequences. Target oligonucleotides are purified from the sample (e.g., to remove extraneous or camouflage oligonucleotides sequences) by washing the sample over a bed of magnetic beads immobilized in a series of microchannels. Each magnetic bead has one or more oligonucleotide probes attached thereto which are complementary to one of the target oligonucleotides that is distinctive to the article of interest. Target oligonucleotides are bound to the oligonucleotide probes of the magnetic beads to form a DNA-probe conjugate. This sequence-specific DNA purification improves downstream amplification efficiency by removing background oligonucleotide sequences (i.e., the target oligonucleotides are purified). The sample is moved across the capture channels using a micropump.

The magnetic beads serve as a medium to carry the target oligonucleotides to an isothermal amplification unit for amplification. The magnetic beads including DNA-probe conjugates are pumped into the isothermal amplification unit and the target oligonucleotides are amplified by isothermal amplification.

The amplified products are pumped through a capture gel suitable for post-amplification injection into a capillary electrophoresis column. The amplified products build up in the capture gel to form a sample plug which can be pumped into a capillary electrophoresis column for separation and detection. The sample plug is released (e.g., thermally released) and injected into a capillary electrophoresis column for target oligonucleotide separation and detection of each of the target oligonucleotide sequences that is present. Oligonucleotide separation is performed by the application of an electric field according to standard capillary electrophoresis techniques known in the art. A laser with an optical detector system is employed to detect the presence of the target oligonucleotides included in the sample plug. Capillary electrophoresis and DNA detection techniques are described in Schwartz and Guttman. Separation of DNA by capillary electrophoresis. Beckman, 1995.

A target sequence profile is generated according to the detected target oligonucleotides from the sample. The target sequence profile includes only the detected target oligonucleotide sequences, but excludes the non-target (camouflage) oligonucleotide sequences. Profile data is either transmitted to a remote server or stored on a local disk for comparison with a library of known sequence profiles to determine if the sample has been provided from an authentic article or not.

### Example 3

### An Integrated Microsystem and Method for Rapid in-Field Detection of a Distinctive Marker

A sample of a DNA marker of interest is provided and transferred to the integrated microsystem device for analysis. The sample includes one or more oligonucleotide sequences. The oligonucleotide sequences may include one or more target oligonucleotide sequences of a distinctive marker and one or more non-target oligonucleotide sequences as camouflage or carrier decoy sequences.

The integrated microsystem device includes a reaction chamber, a relief chamber, a reaction chamber plunger, a temperature control module, an optical detection module, and a data analysis module. The integrated microsystem device utilizes a mechanical fluorescence background displacement technique, which employs the following principle. An array of immobilized oligonucleotide capture probes is disposed in the reaction chamber. A solution comprising fluorescently labeled reporter oligonucleotide probes that are complementary to the immobilized oligonucleotide capture probes is also provided in the reaction chamber. Amplicons generated in the reaction chamber that are complementary to the reporter oligonucleotide probes compete with the capture probes for binding with the reporter oligonucleotide probes. That is, the reporter probes are each also complementary to a specific target oligonucleotide sequence. Thus, as the number of a particular amplicons (i.e., target oligonucleotide sequences) increases, the fluorescent signal detected in the corresponding immobilized oligonucleotide capture probe decreases because fewer reporter oligonucleotide probes bind the corresponding capture probe. This allows simultaneous qualitative and quantitative detection of target nucleic acid sequences included in the oligonucleotide sequences of the sample.

According to the present example, prior to the introduction of the sample to the reaction chamber, the reporter probes can freely bind to the capture probes and a baseline fluorescent signal can be acquired. Fluorescent signals are acquired by actuating the reaction chamber plunger to press the capture probe array against a side wall of the reaction chamber, at which point the optical detection module can acquire a fluorescent signal. When the reaction chamber plunger is not actuated, the capture probe array is not pressed against the wall of the reaction chamber, and thus the fluorescent signal is not detected by the optical detection module. Actuating the reaction chamber plunger also displaces the reaction mixture holding unbound reporter probes so that only bound reporter probes (bound to the capture probes) are detected.

The provided sample is introduced into the reaction chamber and the target oligonucleotide sequences are amplified by PCR. To detect a fluorescent signal pattern over time, the system is configured to sequentially capture fluorescent signal images after each PCR amplification cycle. The temperature control module is configured to automatically heat and cool the reaction chamber for PCR amplification and the reaction chamber plunger is actuated for fluorescent signal capture after each PCR amplification cycle is completed. However, the reaction mixture may emit a background fluorescent signal which could disrupt capture of the fluorescent signal from the capture probe array. Therefore, when the reaction chamber plunger is actuated, it compresses the reaction chamber and thereby displaces the reaction mixture into the relief chamber, thus removing the reaction mixture from the reaction chamber, thus substantially eliminating the background fluorescent signal. After fluorescent signal capture from the capture probe array, the reaction chamber plunger retracts, the reaction chamber relaxes, and the reaction mixture flows from the relief chamber back into the reaction chamber. This process is repeated during each PCR amplification cycle.

The data analysis module is configured to gather optical data from the optical detection module to both qualitatively determine the presence of one or more target oligonucleotide sequences in the ample and/or quantitatively determine the rate of PCR amplification (i.e., the number of amplicons produced) for the one or more target oligonucleotide sequences. A target sequence profile is generated by the data analysis module and the data is either transmitted to a remote server or stored on a local disk for comparison with a library of known sequence profiles to determine if the sample has been provided from an authentic article or not.

### Example 4

### An Integrated Microsystem and Method for Rapid in-Field Detection of a Distinctive Marker

The integrated microsystem described in example 3 may be modified to eliminate thermal cycling by employing isothermal amplification. Therefore, the temperature control module may be omitted, and the rate of image capture may be increased to accommodate the faster cycling time for isothermal amplification.

The methods and systems of the present invention can also be used to verify the authenticity of security marker DNA applied to any item subject to counterfeiting or theft. These items include, for instance, and without limitation, printed materials, bank notes, financial instruments, coins, labels, logos, badges, fabrics, textiles and other commodities, microchips and other electronic items, such as desktop computers, laptops, tablets and smartphones, to name but a few.

The disclosures of each of the references, patents and published patent applications disclosed in this application are each hereby incorporated by reference herein in their entireties.

Having described exemplary embodiments of the present invention, it is further noted that it is readily apparent to those of ordinary skill in the art that various modifications may be made without departing from the spirit and scope of the invention which is defined by the metes and bounds of the appended claims.

What is claimed is:

1. A method of in-field detection of a distinctive marker, comprising:
providing a sample from an article of interest;
analyzing the sample to detect the presence of a distinctive marker, wherein the analysis is performed using an in-field detection instrument, and wherein the in-field detection instrument comprises a microsystem configured to perform sample in-answer out analysis; and
detecting the presence of the distinctive marker in the sample.

2. The method of in-field detection of a distinctive marker of claim 1, wherein the sample from the article of interest further comprises one or more DNA taggants in addition to the distinctive marker, wherein the DNA taggants encode information related to the article of interest; and
detecting the presence of at least one of the DNA taggants in the sample in addition to detecting the presence of the distinctive marker in the sample.

3. The method of in-field detection of a distinctive marker of claim 1, wherein the sample further comprises a sample identifier.

4. The method of in-field detection of a distinctive marker of claim 3, wherein the sample identifier includes an optical reporter, a digital code, a QR code or a bar code.

5. The method of in-field detection of a distinctive marker of claim 4, wherein the optical reporter comprises an upconverting phosphor, a fluorophore, a dye, a stain, or a phosphor.

6. The method of in-field detection of a distinctive marker of claim 1, wherein the distinctive marker comprises a biomolecule or an organic non-biological molecule.

7. The method of in-field detection of a distinctive marker of claim 6, wherein the biomolecule is selected from the group consisting of an amino acid, a peptide, a protein, a lipid, an isoprene, a monosaccharide, a polysaccharide, a fatty acid, a vitamin, a nucleic acid, a protein-DNA complex, and a peptide nucleic acid (PNA).

8. The method of in-field detection of a distinctive marker of claim 7, wherein the nucleic acid comprises DNA.

9. The method of in-field detection of a distinctive marker of claim 8, wherein the distinctive marker comprises one or more unique DNA sequences capable of being copied to produce a unique amplicon profile.

10. The method of in-field detection of a distinctive marker of claim 9, wherein the unique amplicon profile comprises one or more amplicons of varying length.

11. The method of in-field detection of a distinctive marker of claim 1, wherein the in-field detection instrument communicates with a server comprising authenticity data for the article of interest.

12. The method of in-field detection of a distinctive marker of claim 1, wherein the in-field detection instrument is a stand-alone device configured to store and process authenticity data for the article of interest.

13. The method of in-field detection of a distinctive marker of claim 8, wherein the sample is analyzed using PCR, isothermal amplification or nucleic acid hybridization.

14. The method of in-field detection of a distinctive marker of claim 8, wherein the sample is analyzed using next-generation sequencing.

15. The method of in-field detection of a distinctive marker of claim 1, wherein the in-field detection instrument is portable.

16. The method of in-field detection of a distinctive marker of claim 1, wherein the presence of the distinctive marker is detected rapidly.

17. The method of in-field detection of a distinctive marker of claim 1, wherein the presence of the distinctive marker is detected substantially instantaneously.

18. A method of in-field detection of a distinctive marker, comprising:
providing a sample from an article of interest;
analyzing the sample to detect the presence of a distinctive marker, wherein the analysis is performed using an in-field detection instrument, and wherein the in-field detection instrument comprises a microsystem configured to perform sample in-answer out analysis; and
verifying the authenticity of the article of interest by detecting the presence of the distinctive marker in the sample.

19. The method of in-field detection of a distinctive marker of claim 18, wherein the sample further comprises a sample identifier.

20. The method of in-field detection of a distinctive marker of claim 18, wherein the distinctive marker comprises a biomolecule or an organic non-biological molecule.

21. The method of in-field detection of a distinctive marker of claim 20, wherein the biomolecule comprises DNA.

22. The method of in-field detection of a distinctive marker of claim 21, wherein the distinctive marker comprises one or more unique DNA sequences capable of being copied to produce a unique amplicon profile.

23. The method of in-field detection of a distinctive marker of claim 21, wherein the sample is analyzed using PCR, isothermal amplification or nucleic acid hybridization.

24. The method of in-field detection of a distinctive marker of claim 18, wherein the sample is analyzed using an array tube comprising a probe microarray.

25. The method of in-field detection of a distinctive marker of claim 18, wherein the sample is analyzed using capillary electrophoresis.

26. The method of in-field detection of a distinctive marker and claim 18, wherein the sample is analyzed using mechanical fluorescence background displacement.

27. The method of in-field detection of a distinctive marker of claim 26, wherein the sample is amplified by isothermal amplification.

28. A kit for collecting a sample from an article of interest, comprising:
a sample collection unit configured to collect a sample comprising a distinctive marker suitable for analysis in an in-field detection instrument.

29. The kit for collecting a sample from an article of interest of claim 28, wherein the kit further comprises a buffer suitable for extracting the distinctive marker from an article of interest.

30. A device for in-field detection of a distinctive marker, comprising:
an in-field detection instrument comprising a microsystem configured to perform sample in-answer out analysis, wherein the in-field detection instrument is configured to analyze a sample to determine the presence of a distinctive marker in the sample.
